(19) United States
(12) Patent Application Publication
(10) Pub. No.: US 2023/0190748 A1
(43) Pub. Date: Jun. 22, 2023

(54) COMPOSITIONS FOR TREATMENT OF AGED DISEASES

(71) Applicant: GERO PTE. LTD., Singapore (SG)

(72) Inventors: Maksim Nikolaevich Kholin, Moskva (RU); Olga Andreevna Burmistrova, Reutov (RU); Konstantin Avkhachev, Moskva (RU); Petr Olegovich Fedichev, Dolgoprudnyy (RU)

(21) Appl. No.: 17/914,572

(22) PCT Filed: Mar. 26, 2021
(86) PCT No.: PCT/RU2021/050081

§ 371 Date: Sep. 26, 2022

(30) Foreign Application Priority Data
Mar. 27, 2020 (RU) 2020112682

(51) Int. Cl. A61K 31/517; A61P 39/00

(52) U.S. Cl. CPC A61K 31/517; A61P 39/00

(57) ABSTRACT

The present disclosure provides compounds, combinations, methods, and kits for the treatment of aging and age related disorders and related mediums.

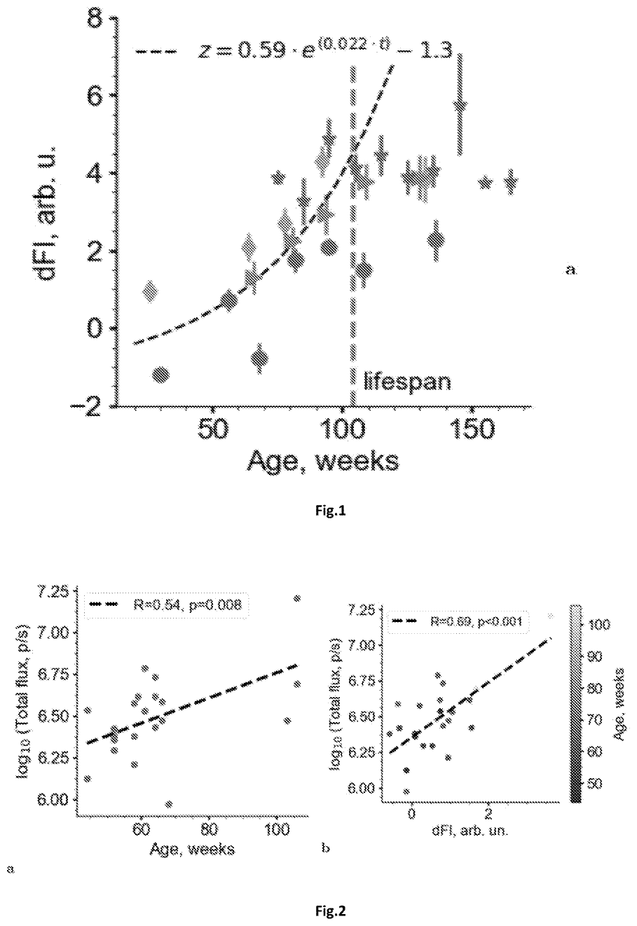

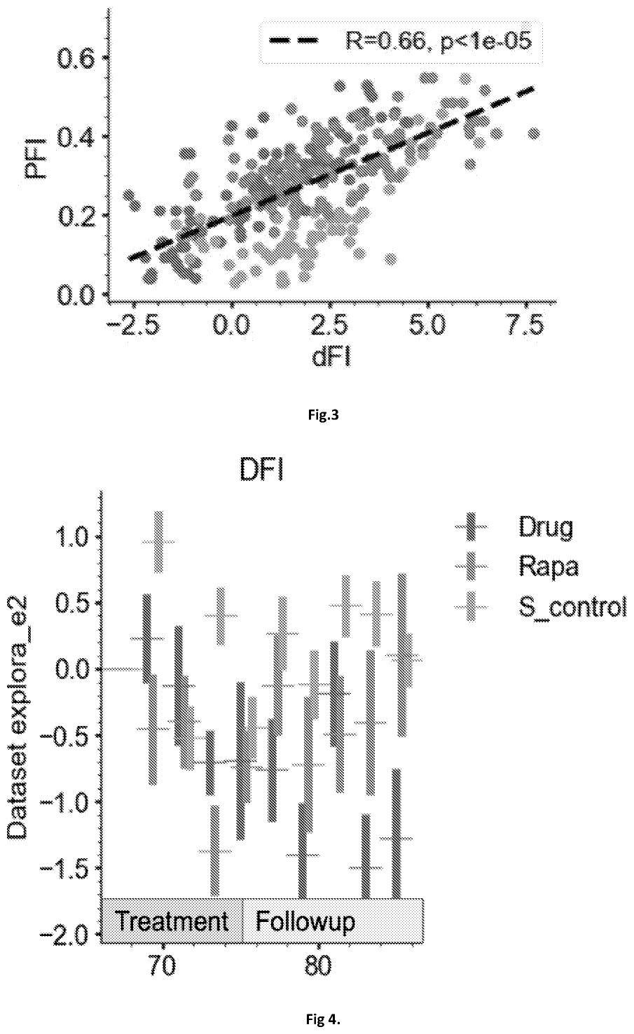

### BACKGROUND

The development of methods, medications, for increasing health span or treating aging or age-related diseases would benefit many. We disclose the anti-aging effect of several compounds and their analogs and combinations described herein as well as related methods.

Epidermal growth factor receptor (EGFR, also known as ErbB-1 or HER-1) inhibitors are medicines that bind to certain parts of the EGFR and slow down or stop cell growth.

EGFR is a protein that is found on the surface of some cells that causes cells to divide when epidermal growth factor binds to it. EGFR is found at abnormally high levels in cancer cells, and EGFR activation appears to be important in tumor growth and progression. Some types of cancers show mutations in their EGFRs, which may cause unregulated cell division through continual or abnormal activation of the EGFR.

EGFR inhibitors can be classified as either:
tyrosine kinase inhibitors (TKI) (eg, erlotinib, gefitinib): these bind to the tyrosine kinase domain in the epidermal growth factor receptor and stop the activity of the EGFR
monoclonal antibodies (eg, cetuximab, necitumumab): these bind to the extracellular component of the EGFR and prevent epidermal growth factor from binding to its own receptor, therefore preventing cell division.

### BRIEF DESCRIPTION OF THE DRAWINGS

The novel features of the invention are set forth with particularity in the appended claims and description. A better understanding of the features and advantages of the present invention will be obtained by reference to the following detailed description that sets forth illustrative embodiments, in which the principles of the invention are utilized, and the accompanying drawings of which:

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising,” are to be construed in an open, inclusive sense, that is, as “including, but not limited to.” In addition, the term “comprising” (and related terms such as “comprise” or “comprises” or “having” or “including”) is not intended to exclude that in other certain

embodiments, for example, an embodiment of any composition of matter, composition, method, or process, or the like, described herein, may “consist of or “consist essentially of the described features. Headings provided herein are for convenience only and do not interpret the scope or meaning of the claimed embodiments.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise. Thus, for example, reference to “a non-human animal” may refer to one or more non-human animals, or a plurality of such animals, and reference to “a cell” or “the cell” includes reference to one or more cells and equivalents thereof (e.g., plurality of cells) known to those skilled in the art, and so forth. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. For example, the use of “about X” shall encompass +/−1%, 2%, 3%, 4%, 5%, 6%, 7%, 8%, 9%, 10%, 11%, 12%, 13%, 14% and 15% of the value X. It should also be noted that the term “or” is generally employed in its sense including “and/or” unless the content clearly dictates otherwise. The term, “at least one,” for example, when referring to at least one compound or to at least one composition, has the same meaning and understanding as the term, “one or more.

Disclosed herein are compounds, compositions and combinations, kits, and methods for anti-aging treatment and related mediums and methods.

Afatinib is approved in a number of markets, including the EU, Japan, Taiwan and Canada under the brand name Giotrif®, in the US under the brand name Gilotrif) and in India under the brand name Xovoltib® for use in patients with distinct types of EGFR mutation-positive NSCLC. Afatinib is also approved in the EU, US and other markets for the treatment of patients with advanced SqCC of the lung whose disease has progressed (on or) after treatment with platinum-based chemotherapy. Afatinib is under regulatory review by health authorities in other countries worldwide.

In some embodiments, GILOTRIF® tablets contain afatinib, a tyrosine kinase inhibitor which is a 4-anilinoquinazoline. In some embodiments, Afatinib is presented as the dimaleate salt, with the chemical name 2-butenamide, N-[4-[(3-chloro-4-fluorophenyl)amino] 7-[[(3S)-tetrahydro-3-furanyl]oxy]-6-quinazolinyl]-4-(dimethylamino)-,(2E)-, (2Z)-2-butenedioate (1:2). In some embodiments, Its structural formula is:

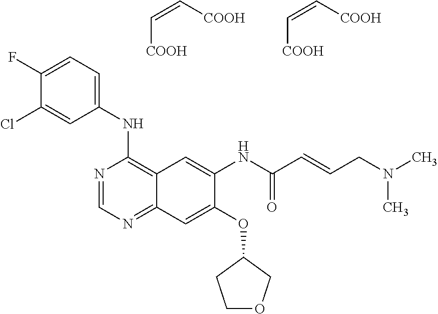 [SMILES: O=C(O)/C=C\C(=O)O ; O=C(O)/C=C\C(=O)O ; CN(C)C/C=C/C(=O)Nc1cc2c(Nc3ccc(F)c(Cl)c3)ncnc2cc1O[C@H]1CCOC1]

In some embodiments, Afatinib dimaleate is a white to brownish yellow powder, water soluble and hygroscopic, with an empirical formula of C32H33ClFN5O11, and a molecular weight of 718.1 g/mol. Afatinib tablets for oral administration are available in 40 mg, 30 mg, or 20 mg of afatinib (equivalent to 59.12 mg, 44.34 mg, or 29.56 mg afatinib dimaleate, respectively). In some embodiments, The inactive ingredients of tablets with Afatinib are the following: Tablet Core: lactose monohydrate, microcrystalline cellulose, crospovidone, colloidal silicon dioxide, magnesium stearate and Coating: hypromellose, polyethylene glycol, titanium dioxide, talc, polysorbate 80, FD&C Blue No. 2 (40 mg and 30 mg tablets only).

It is also known in the field that the effect of lifespan extension goes together with other anti-aging effects, such as, but not limited to healthspan extension, rejuvenation, prevention and treatment of diverse age-related diseases, disorders and declines (including but not limited to Alzheimer's, Parkinson's, and Huntington's diseases, cardiovascular disease, renal failure, muscle wasting [cachexia], osteopenia or osteoporosis, obesity, insulin resistance or diabetes, and diverse adult-onset cancers).

### Definitions

The term “subject,” as used herein, generally refers to an animal, such as a mammalian species (e.g., mouse or human) or avian (i.e., bird) species, nematode (e.g., C. elegans), or other organism, such as a plant. More specifically, the subject can be a vertebrate, e.g., a mammal such as a mouse, a primate, a simian or a human. Animals include, but are not limited to, farm animals, sport animals, and pets. A subject can be a healthy individual, an individual that has or is suspected of having a disease or a predisposition to the disease, or an individual that is in need of therapy or suspected of needing therapy, or an aged or frail individual. A subject can be any human being.

In some embodiments, by treating or preventing an age-related disease or disorder, any anti-aging treatment is meant to include (but is not limited to) treatments leading to prevention, amelioration or lessening the effects of aging, decreasing or delaying an increase in the biological age, slowing rate of aging; treatment, prevention, amelioration and lessening the effects of frailty or at least one of aging related diseases and conditions or declines or slowing down the progression of such decline (including but not limited to those indicated in Table 1, “Declines”), condition or disease, increasing health span or lifespan, rejuvenation, increasing stress resistance or resilience, increasing rate or other enhancement of recovery after surgery, radiotherapy, disease and/or any other stress, decreasing all-causes or multiple causes of mortality risks or mortality risks related to at least one or at least two of age related diseases or conditions or delaying in increase of such risks, decreasing morbidity risks. The treatment leading to the modulating at least one of biomarkers of aging into more youthful state or slowing down its change into “elder” state is also regarded to be an anti-aging treatment, including but not limited to biomarkers of aging which are visible signs of aging, such as wrinkles, grey hairs etc. In some embodiments, an age-related disease or disorder is selected from: atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis, urinary incontinence etc. Aging-related changes in any parameter or physiological metric are also regarded as age-related conditions, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.). Aging related change in any parameter of organism is also regarded as an aging related condition, including but not limited to aging related change in at least one of the parameter selected from the Table 1 “Declines”.

In some embodiments of this invention “aged subject” is understood as a human being of chronological age (or in some embodiments, of biological age) of 30 years or older, 35 years or older, 40 years or older, 45 years or older, 50 years or older, 55 years or older, 60 years or older, 65 years or older, 70 years or older, 75 years or older, 80 years or older, 85 years or older, 90 years or older, 95 years or older. In some embodiments of this invention “aged subject” is understood as a frail human (or other animal).

In some embodiments, the term “small molecule” means an individual compound with molecular weight less than about 2000 daltons, usually less than about 1500 daltons, more usually less than about 750 daltons, preferably less than about 500 daltons, although molecules larger than 2000 daltons in size will also be included herein.

In some embodiments, the term EGFR inhibitor comprises any one molecule selected from the group: AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, In case any of the above is not a molecule but the composition or formulation, in some embodiments of this invention the term EGFR inhibitor comprises all such composition or formulation, and in some of this invention the term EGFR inhibitor comprises Active Pharmaceutical Ingredient of such composition or formulation inhibiting EGFR.

### Pharmaceutical Compositions

Disclosed herein is a new anti-aging use of compound selected from EGFR inhibitors, including but not limited to AC480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190. In case any of the above is not a molecule but the composition or formulation, in some embodiments and their analogs.

Disclosed herein is a new combination of at least two compounds selected from EGFR inhibitors, including but not limited to AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 and an anti-aging use of such combination.

In some embodiments, this disclosure provides a medication or pharmaceutical composition comprising compounds selected from from EGFR inhibitors, including but not limited to AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their structural or functional analogs or prodrugs. In some embodiments, some and in some other embodiments—all subsequent mentions of these drugs are intended to include their structural and functional analogs or prodrugs.

COMBINATION WITH OTHER ANTI-AGING DRUGS: ANY compound of this disclosure can be used with other anti-aging drugs or interventions which will increase anti-aging effects, Some possible geroprotectors include melatonin,[4] carnosine,[5] metformin,[6] nicotinamide mononucleotide (NMN)[7] and delta sleep-inducing peptide.[8] NAD+, bASIS™, In some embodiments such pharmaceutical composition is for use as an anti-aging medication or for use for the anti-aging treatment or rejuvenation.

In some instances, the pharmaceutical composition described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. Where the pharmaceutical composition described herein is administered by infusion, it can be dispensed, for example, with an infusion bottle containing sterile pharmaceutical grade water or saline. Where the pharmaceutical composition described herein is administered by injection, an ampule of sterile water for injection or saline can be provided so that the enzyme or enzyme and antioxidant and the carrier can be mixed prior to administration. One of the many possible forms of this invention can be a Lyophilized Concentrate for Intravenous (IV) Injection.

The amount of pharmaceutical composition described herein that is effective for treating a corresponding disease or condition can be determined using standard clinical or pharmacokinetic techniques known to those with skill in the art. In addition, in vitro or in vivo assays can optionally be employed to help identify optimal dosage ranges. The precise dose to be employed can also depend on the route of administration, the disease or condition, the seriousness of the corresponding disease or condition being treated, as well as various physical factors related to the individual being treated, and can be decided according to the judgment of a health-care practitioner. For example, any agent or composition of this disclosure, in an amount ranging from about 0.05 μg/kg to about 100 mg/kg of a patient's body weight, or 0.01 to about 1000 mg/kg of total body weight per day, or from about 0.1 to about 100 mg/kg of total body weight per day, or from about 0.5 to about 15 mg/kg of total body weight per day, or from about 1 mg/kg to about 50 mg/kg of total body weight, which may be administered in one or multiple doses per day or per week or per month or per 6 months or per year or per 3 years or per 8 years or per 12 years or once in a lifetime. In some embodiments, this invention is a pharmaceutical composition and formulation, comprising amount of the agent needed for a subject with weight of 50 kg or in some embodiments 75 kg or in some embodiments 90 kg to provide the amount of the agent described above and at least one pharmaceutically acceptable excipient. Equivalent dosages can be administered over various time periods including, but not limited to, about every 2 hours, about every 4 hours, about every 8 hours, about every 12 hours, about every 24 hours, about every 36 hours, about every 48 hours, about every 72 hours, about every week, about every two weeks, about every three weeks, about every month, and about every two months or every 6 months or every year or every 3 years or every 8 years or every 12 years or once in a lifetime or daily lifelong or as decided by practitioner or patient. The number and frequency of dosages corresponding to a completed course of therapy can be determined according to the judgment of a health-care practitioner. In some embodiments, in case of toxicity or adverse effects the administration can be suspended or dosage decreased until the toxicity or adverse effects disappear and then the administration and/or dosage can be resumed on the previous level.

In some embodiments, the pharmaceutical composition and formulations described herein are administered to a subject by any suitable administration route, including but not limited to, parenteral (e.g., intravenous, subcutaneous, intramuscular), intradermal, intraperitoneal, subcutaneous, intranasal, epidural, sublingual, intravaginal, rectal, by inhalation, topical intracerebral, oral, intranasal, buccal, rectal, or transdermal administration routes. For example, in some instances, the pharmaceutical composition described herein is administered locally. This is achieved, for example, by topical application (including but not limited when prodrugs that require esterase activation after uptake may be applicable topically), local infusion during surgery, by injection, by means of a catheter, by means of a suppository or enema, or by means of an implant, the implant being of a porous, non-porous, or gelatinous material, including membranes, such as sialastic membranes, or fibers. In some situations, the pharmaceutical composition described herein is introduced into the central nervous system, circulatory system or gastrointestinal tract by any suitable route, including intraventricular, intrathecal injection, paraspinal injection, epidural injection, enema, and by injection adjacent to a peripheral nerve. Pulmonary administration can also be employed, e.g., by use of an inhaler or nebulizer, and formulation with an aerosolizing agent, or via perfusion in a fluorocarbon or synthetic pulmonary surfactant.

In some embodiments, the pharmaceutical formulations include, but are not limited to, aqueous fluid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some embodiments, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some embodiments, the pharmaceutical formulations further include diluents which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Methods and Uses

Described herein are methods of treating or preventing an age-related disease or disorder or other anti-aging treatment comprising administering to a subject in need thereof an agent or pharmaceutical composition described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In some embodiments, the compounds and combinations of this disclosure are useful for changing selected biomarkers related to aging or mortality risks into a younger state and thus reducing the risks of mortality, including but not limited to biomarkers described in Table 2, https://www.ncbi.nlm.nih.gov/pmc/articles/PMC5611985/ or on the website http://mortalitypredictors.org and the publications cited there on blood predictors of mortality or in any other source.

Every web link cited in this application, in case of inaccessibility can be retrieved via https://web.archive.org or similar internet archive services.

### Kit

In some embodiments this invention is a kit, comprising an compound or agent, disclosed or described in this disclosure, or composition disclosed in this application or its functional or structural analog or prodrug and the notice, description or instruction for its use for anti-aging treatment, optionally comprising at least the medication labeling information.

In some embodiments this invention is a kit, comprising an compound or agent, disclosed or described in this disclosure, or composition disclosed in this application or its functional or structural analog or prodrug and the notice, description or instruction for its use by human or by other animal subject in a dosage and regimen to maintain concentration of such compound in blood of such subject in 1 μM level at 30% of time, or from 0.5 to 5 μM level at from around 1% of time to around 100% of time, or from 0.5 to 5 μM level at from around 10% of time to around 50% of time, or from 0.05 to 5 μM level or at least one other level described in this disclosure.

The kit of this invention can be made of any material such as paper, plastic, steel, glass, etc, including but not limited to materials used in the art for kits for medications.

In some embodiments, this invention is an compound (agent) of this disclosure with the mentioned notice, description or instruction attached to such agent or imprinted or drawn or in any other way displayed on such agent or in any other way associated with such agent (e.g. in machine readable form). One of the primary purposes of some aspects of his invention is to provide medication for anti-aging treatment.

In some embodiments the contents and appearance of such notice, description or instruction is regulated by the respective national or international rules regarding labeling of medication, incorporated here by reference or such notice, description or instruction comprise at least part or optionally most of the or optionally all the information required by applicable labeling regulations.

The notice, description or instruction, including but not limited to labeling means (e.g., treatment and/or operation guidelines) can be provided in any form that conveys the requisite information. Instruction means can be audio, for example spoken word, recorded in analog or digital form (e.g., audio recording), or received and/or transmitted in analog or digital form (e.g., by telephone, conference call, or audio signal transmitted over a network). Such information can also be visual or video, for example hard-copy (e.g., as a manual, recorded medium, booklet, leaflet, book and the like) or soft-copy (e.g., recorded in analog or digital form as a file recorded on an magnit, electronic, optical, or computer readable medium such as a DVD, disk drive, CD-ROM and the like). Additionally, instruction means can be interactive or real-time (e.g., a teleconference or internet chat or chat bot).

Some kits or agents of this invention can include printed or made in any other way instructions to inform the user of the steps required to properly use it.

In some embodiments, agents and kits, of this invention include a label configured to be coupled to respective agent and kits of this invention. The label includes a first surface and a second surface. In some embodiments, the first surface can be coupled to an outer surface of agents and kits of this invention. In some embodiments, for example, the first surface can include an adhesive. The second surface can include include a textual indicia, such as, for example, a description of the of agents and kits of this invention, a mark indicating its manufacturer or distributor and/or an instruction associated with the use of such of agents and kits of this invention. The label can further include an electronic circuit system configured to output an electronic signal. In some embodiments, the electronic signal can include an instruction associated with the use of the mediums, kits, devices or agents of this invention.

In some embodiments the instruction is an instruction for use as anti-aging medication. In some embodiments the instruction is an instruction for use as medication for a treating or preventing an age-related disease or disorder. In some embodiments, the notice, description or instruction, including but not limited to labeling can be shown on the lenses, computer glasses, transmitted via brain computer interface or by any other means or can be encoded by the Quick Response Code or any other machine readable form.

The notice, description or instruction, including but not limited to labeling, can be implemented in digital electronic circuitry, or in computer firmware, hardware, software, or in combinations thereof. The implementation can be as a computer program product, e.g., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be recorded in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site or several sites.

The notice, description or instruction, including but not limited to labeling can be performed by one or more programmable processors executing a computer program to perform functions of the invention by operating on input data and generating output. It can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

In some embodiments this invention is a method of anti-aging treatment, comprising administering by the subject at least one of the compositions, agents or molecules described in this disclosure, in therapeutically effective amount. In some embodiments this invention is a method of anti-aging treatment, comprising administering to the subject an effective amount of molecule selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics in therapeutically effective amount.

The dosage levels and mode of administration will be dependent on a variety of factors such as the treatment used, the device, the active agent, the context of use (e.g., the patient to be treated), and the like. Optimization of modes of administration, dosage levels, and adjustment of protocols, including monitoring systems to assess effectiveness are routine matters well within ordinary skill.

Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein, to which the reader is referred.

The compounds of this invention are known in the art as well as the process of its manufacturing. The compounds of this invention can also be acquired from commercial sources.

In some embodiments, the one or two or more biomarkers (optionally—with associated measurement units in plasma) which will be changed into more youthful state as a result of administration of compound or combination of this invention is a biological age characteristic. In some embodiments, biological age characterizes the health status of the subject.

In some embodiments, compounds, combinations are administered by aged subject.

In some embodiments, the biological age determination approach is described in prior art, including but not limited to any of the following publications https://www.ncbi.nlm.nih.gov/pmc/articles/PMC5514388/. https://www.ncbi.nlm.nih.gov/pmc/articles/PMC4931851/, https://www.ncbi.nlm.nih.gov/pmc/articles/PMC5514388/ and corresponding references related to blood based biological age determination.

In some embodiments, the biological age is understood as the distance measured along a continuous trajectory consisting of distinct phases, each corresponding to subsequent human life stages as described in more details in https://www.biorxiv.org/content/biorxiv/early/2017/09/09/186569.full.pdf

In some embodiments, this at least one of the compound, composition or combination of this disclosure reduces mortality risk or high mortality risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, mortality risk is a risk of dying from age related condition or disease. In some embodiments, mortality risk is all cause mortality risk. Non limiting examples of biomarkers of mortality and its critical volumes are described in prior art, including but not limited to https://www.ncbi.nlm.nih.gov/pmc/articles/PMC4899173/, https://www.ncbi.nlm.nih.gov/pmc/articles/PMC4454670/, https://www.ncbi.nlm.nih.gov/pmc/articles/PMC5334528/ Maximus Peto, Carlos De la Guardia, Ksenia Winslow, Andrew Ho, Kristen Fortney, & Eric Morgen. “Mortalitypredictors.org, a manually curated database of published biomarkers of human all-cause mortality. Aging, 2017.

In some embodiments, at least one of the compound, composition or combination of this disclosure reduces morbidity risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, morbidity risk is a risk of acquiring an age related condition or disease.

In some embodiments, this invention is at least one of the compound, composition or combination of this disclosure useful for treatment and prevention of disease selected from the group: type 2 diabetes, age-related cardiovascular diseases, including but not limited to ischaemic heart disease and stroke, metabolic syndrome, COPD, Alzheimer's disease etc., including but not limited those mentioned in this disclosure or at least one of the aging related declines.

In some embodiments this invention is a method, including but not limited to method of testing of efficacy of the compound, composition or combination of this disclosure, comprising the checking in the patient treated by such compound, composition or combination of this disclosure at least one of the following: biological age of the patient, at least one aging biomarker, at least one age related deficit or disease, at least one of rejuvenation marker, frailty, health span or life span or in any other reasonable method to check the efficacy of anti-aging treatment.

An age-related pathology that can be treated by compound selected from the group: EGFR inhibitor, AC-480, AEE-788, AEE788, afatinib, ARRY-334543, AST 1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 may include any disease or condition which is fully or partially mediated by the induction or maintenance of a non-proliferating or senescent state in a cell or a population of cells in a subject. Non-limiting examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, and others. A method of identifying an age-related disease or condition as described herein may include detecting the presence of senescent cells.

In some embodiments, a senescence-associated pathology that can be treated by compound selected from the group: EGFR inhibitor, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTINAG 1478, VANDETANIB, VARLITINIB, VATALANIB, XL-647, ZD-4190 may include any disease or condition which is fully or partially mediated by the induction or maintenance of a non-proliferating or senescent state in a cell or a population of cells in a subject. Non-limiting examples include cardiovascular diseases such as angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune diseases such as herniated intervertebral disc, inflammatory bowel disease, kyphosis, oral mucositis, lupus, interstitial cystitis, scleroderma, and alopecia; neurodegenerative diseases such as dementia, Huntington's disease, motor neuron dysfunction, age-related memory decline, and depression/mood disorders; metabolic diseases such as diabetic ulcer and metabolic syndrome; pulmonary diseases such as age-related loss of pulmonary function, asthma, bronchiectasis, cystic fibrosis, emphysema, and age-associated sleep apnea; gastrointestinal diseases such as Barrett's esophagus; age-related disorders such as liver fibrosis, muscle fatigue, oral submucosa fibrosis, pancreatic fibrosis, benign prostatic hyperplasia (BPH), and age-related sleep disorders; reproductive disorders such as menopause (male and female), egg supply (female), sperm viability (male), fertility (male and female, sex drive, and erectile function and arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, hyperpigmentation, immunobullous dermatosis, nevi, pemphigoid, pemphigus, pruritis, psoriasis, rashes, reactive neutrophilic dermatosis, rhytides, and urticarial: and other diseases such as diabetic wound healing, post-transplant kidney fibrosis, and carotid thrombosis. In some embodiments, a method is provided for treating a senescence associated disease or disorder in a subject comprising administering to the subject a compound or combination, combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 wherein the senescence associated disease or disorder is not a cancer, wherein such compound or combination is administered during a treatment course of 1-7 days every 0.5-12 months; provided that if the senescence associated disease or disorder is a senescence associated metabolic disorder, such compound or combination is administered during a treatment course of 1-7 days every 4-12 months. In certain embodiments, such compound or combination is administered once every 0.5-12 months; provided that if the senescence associated disease or disorder is a senescence associated metabolic disorder, such compound or combination is administered once every 4-12 months. In other certain embodiments, the aging related disease or senescent cell-associated disease or disorder is a cardiovascular disease or disorder, inflammatory disease or disorder, a pulmonary disease or disorder, a neurological disease or disorder. In a specific embodiment, the cardiovascular disease or disorder is atherosclerosis. In another specific embodiment, the inflammatory disease or disorder is osteoarthritis. In another specific embodiment, the pulmonary disease or disorder is idiopathic pulmonary fibrosis or chronic obstructive pulmonary disease. In another specific embodiment, the neurological disease or disorder is selected from mild cognitive impairment; motor neuron dysfunction; Alzheimer's disease; Parkinson's disease; and macular degeneration. In another specific embodiment, the senescence associated metabolic disease or disorder is selected from diabetes, metabolic syndrome, and obesity. In another specific embodiment, the aging related disease or senescence-associated disease or disorder is a dermatological disease or disorder is selected from eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus.

In some embodiments, a method is provided for treating a senescence-associated metabolic disease or disorder in a subject comprising administering to the subject a compound or combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, wherein such compound or combination is administered during a treatment course of 1-7 days every 4-12 months, and wherein the metabolic disease or disorder is selected from diabetes, metabolic syndrome, and obesity. In a specific embodiment, such compound or combination is administered once every 4-12 months. In another specific embodiment, the senescent cell is selected from a senescent fibroblast, a senescent pre-adipocyte, a senescent epithelial cell, a senescent chondrocyte, a senescent neuron, and a senescent endothelial cell. In another specific embodiment, the senescent cell is a senescent pre-adipocyte.

In one embodiment, a method is provided for treating, reducing the likelihood of occurrence of, or delaying onset of age related disease or decline or a senescent cell-associated disease or disorder in a subject who has an age related disease or decline a senescent cell-associated disease or disorder or who has at least one predisposing factor for developing the senescent cell-associated disease or disorder, comprising administering to the subject a compound or combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, thereby promoting death of the senescent cell, wherein the senescent cell-associated disease or disorder is a cardiovascular disease or disorder, inflammatory disease or disorder, a pulmonary disease or disorder, a neurological disease or disorder, with the proviso that if the subject has a cancer, and such compound is not a primary therapy for treating the cancer, and wherein the such compound or composition is administered once every 0.5-12 months. In another embodiment, a method is provided for treating, reducing the likelihood of occurrence of, or delaying onset of a senescent cell-associated disease or disorder in a subject who has a senescent cell-associated disease or disorder or who has at least one predisposing factor for developing the senescent cell-associated disease or disorder, comprising administering to the subject a seno lytic combination comprising (a) a first agent that alters either one or both of a cell survival signaling pathway and an

In another embodiment, a method is provided for killing a senescent cell comprising contacting the senescent cell and a a compound or combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, thereby promoting death of the senescent cell, wherein the senescence cell is present in a subject who has a senescent cell-associated disease or disorder or who has at least one predisposing factor for developing the senescent cell-associated disease or disorder, with the proviso that if the subject has a cancer, and such compound or is not a primary therapy for treating the cancer, wherein the senescent cell-associated disease or disorder is a cardiovascular disease or disorder, inflammatory disease or disorder, a pulmonary disease or disorder, a neurological disease or disorder, wherein the combination is administered once every 0.5-12 months.

In certain embodiments of the methods described above and herein, the senescent cell-associated disease or disorder is selected from atherosclerosis: osteoarthritis; idiopathic pulmonary fibrosis; chronic obstructive pulmonary disease: mild cognitive impairment; motor neuron dysfunction; Alzheimer's disease; Parkinson's disease; and macular degeneration.

In another embodiment, a method is provided for killing a senescent cell comprising contacting the senescent cell and compound or combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, thereby promoting death of the senescent cell, wherein the senescence cell is present in a subject who has a senescent cell-associated disease or disorder or who has at least one predisposing factor for developing the senescent cell-associated disease or disorder, wherein the senescent cell-associated disease or disorder is a metabolic disorder selected from diabetes, metabolic syndrome, and obesity, and wherein the combination is administered once every 4-12 months.

In another embodiment, a method is provided for treating or reducing the likelihood of occurrence of atherosclerosis in a subject who has atherosclerosis or who has at least one predisposing factor for developing atherosclerosis, comprising administering to the subject a compound or combination, comprising such compound, modulating EGFR, including but not limited to EGFR inhibitor, small molecule EGFR inhibitor, EGFR antibody, EGFR humanized antibody, AC480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, thereby promoting death of the senescent cell, wherein the combination is administered once every 0.5-12 months, and wherein the first and second agents are different.

In certain embodiments of the methods described above and herein, the senescent cell is selected from a senescent fibroblast, a senescent pic-adipocyte, a senescent epithelial cell, a senescent chondrocyte, a senescent neuron, a senescent smooth muscle cell, a senescent mesenchymal cell, a senescent macrophage, and a senescent endothelial cell. In certain specific embodiments, the senescent cell is a senescent pre-adipocyte.

In some embodiments this invention is a method, including but not limited to method of testing of efficacy of compound, composition or combination of this disclosure, comprising the checking in the subject treated by such therapy at least one of the following: checking biological age of the patient, at least one aging biomarker, at least one age related deficit or disease, at least one of rejuvenation marker, frailty, health span or life span, or any other marker or parameter reasonable for checking in testing of anti-aging therapy efficacy.

In some embodiments, this invention is a method of anti-aging treatment, comprising administering by subject at least one compound, composition or combination of this disclosure, and repeating administration in case biological age increased for more than 1 year, more than 3 years, more than 8 years, more than 10 years. In some embodiments the dosage and regimen for implementing disclosed method is defined to keep biological age at the reasonably minimum level, or close to the age that subject had at the time of first treatment.

In some embodiments checking of efficacy of compound, composition or combination of this disclosure and measurement of markers or symptoms of related diseases or conditions is conducted in 1 month after the infusion of treated plasma or administration of therapy in therapeutically effective amount, in 3 months, in 6 months, in 12 months, in 18 months, in 24 months or in 36 months after such infusion, or in around such date, or in date reasonably defined by the practitioner based on the parameter being measured and other factors known to the expert in the field.

Any of the compounds and combinations of this disclosure can be provided in formulations defined by the expert skilled in the art, including but not limited those known in the art and those suggested in the examples.

In some embodiments, compounds selected from EGFR inhibitor, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their structural or functional analogs or prodrugs and other agents of this disclosure to be useful for anti-aging or treatment of age related disease or other disease indicated in this disclosure shall be administered in the same dosages as such agents are effective in their primary indication (Primary indication effective dosage or PIED), which is known in the art. In some embodiments, the dosage for the agent of this disclosure should be at least 100 times less or least 50 times less or at least 10 times less or at least 5 times less, or at least 2 times less, or at least 50% less, or at least 25% less, or at least 10% less, or at least 5 times more, or at least 2 times more, or at least 50% more, or at least 25% more, or at least 10% more, or at least 10 times more, or at least 100 times more than PIED to have anti-aging therapeutic effect. In some embodiments, to have anti-aging therapeutic effect such agent shall be administered at maximum tolerated dose.

In some embodiments, the dosage for the agent of this disclosure, including but not limited to EGFR inhibitor, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 should be about at least 1000 times less at least 100 times less or least 50 times less or at least 10 times less or at least 5 times less, or at least 2 times less, or at least 50% less, or at least 25% less, or at least 10% less, than maximum tolerated dose to have anti-aging therapeutic effect or to be effective against frailty, aging related disease or condition.

In some embodiments this invention is a tangible medium comprising a computer program, which, when executed, causes a medium to perform a method comprising: attribution to the information about a subject an information about an anti-aging treatment related to compound, composition or combination of this disclosure, optionally further comprising attributing to the information about patient before or after or before and after the treatment to information about checking of at least one selected from the group: biological age of the patient, at least one aging biomarker, at least one age related deficit or disease or its symptom, at least one of rejuvenation marker, frailty, health span or life span.

A prophetic example of such tangible medium could be a APPLE™ 2014 MACBOOK AIR™ 13″ Intel™ i5 with Microsoft™ Excel™ installed and executed on it, wherein to patient with name John Junior Smith (born 2 Jan. 1937) the information about using compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics in therapeutically effective amount is attributed in the sense that is logically linked as an information in Excel table (in this example attribution is realized as placing the information about treatment by such drug in the same line in the file with the name and ID of the patient to whom such treatment is prescribed) and allows easy finding of patients to whom such treatment is prescribed and other processing of such information.

| patient | | | | Treatment |
|---|---|---|---|---|
| ID | Name | Date of birth | diagnosis | prescribed |
| 28282839 | John Junior | 2 Jan. 1937 | Before treatment: moderate frailty, | Afatinib |
| | Smith | | moderate cognitive decline, hand grip | |
| | | | strength −21.3 kg and other age related | |
| | | | deficites | |
| 28282838 | peter Junior | 2 Jan. 1937 | Before treatment: covid-19, influenza, | Afatinib |
| | Smith | | pulmonary fibrosis | |
| 28282837 | Val | 2 Jan. 1937 | Before treatment: moderate frailty, | gefitinib |
| | Smith | | moderate cognitive decline, hand grip | |
| | | | strength −21.3 kg and other age related | |
| | | | deficites | |
| 28282836 | Robert | 2 Jan. 1937 | Before treatment: covid-19, influenza, | erlotinib |
| | Smith | | pulmonary fibrosis | |
| 28282835 | John Junior | 2 Jan. 1937 | Before treatment: moderate frailty, | lapatinib |
| | Smith | | moderate cognitive decline, hand grip | |
| | | | strength −21.3 kg and other age related | |
| | | | deficites | |
| 28282834 | Donald | 2 Jan. 1937 | Before treatment: covid-19, influenza, | cetuximab |
| | Junior | | pulmonary fibrosis | |
| | Smith | | | |

Processors suitable for the execution of a computer program related to this invention include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

In some embodiments this invention is a tangible medium comprising a computer program, which, when executed, causes a device to perform a method comprising: attribution to the information regarding compound, composition or combination of this disclosure, an information about anti-aging treatment.

As an example of such attribution the excel file on the same computer as described above or Internet site hosted on server that displays the following can be suggested

| | Agent/Mechanism of | |
|---|---|---|
| Name | Action/Mode of action | Indication |
| ASD1 | Afatinib | anti-aging, anti-frailty, amelioration of moderate |
| | | cognitive decline, amelioration hand grip strength |
| | | lose and amelioration of other age related deficits |
| GER23 | gefitinib | anti-aging, anti-frailty, amelioration of moderate |
| | | cognitive decline, amelioration hand grip strength |
| | | lose and amelioration of other age related deficits |
| GER56 | erlotinib | anti-aging, anti-frailty, amelioration of moderate |
| | | cognitive decline, amelioration hand grip strength |
| | | lose and amelioration of other age related deficits |
| FDS89 | lapatinib | anti-aging, anti-frailty, amelioration of moderate |
| | | cognitive decline, amelioration hand grip strength |
| | | lose and amelioration of other age related deficits |

In some embodiments this invention is a method, method comprising: attribution to the information about a subject an information about an anti-aging treatment related to compound, composition or combination of this disclosure, optionally further comprising attributing to the information about patient before or after or before and after the treatment to information about checking of at least one selected from the group: biological age of the patient, at least one aging biomarker, at least one age related deficit or disease, at least one of rejuvenation marker, frailty, health span or life span. In some embodiments such method is a computer implemented method.

In some embodiments this invention is a method, the method of this invention, comprising attribution of information executed on the medium of this invention and described in corresponding part of this disclosure related to such medium.

In some embodiments this invention is a tangible medium or computer system or processor, comprising a computer program, which, when executed, causes a medium to perform a method comprising attribution of information described in this disclosure.

In some embodiments this invention is an apparatus to execute method described in this disclosure, the apparatus comprising the processor comprising the tangible medium described in this disclosure

In some embodiments, term “Afatinib” is used interchangeably with any one of EGFR inhibitors.

In some embodiments, dose for anti-aging effect of Afatinib is 500 mg (one tablet) 3-4 times a day. In some embodiment, dose of Afatinib is from about 50 mg to about 4000 mg. In some embodiment, dose of Afatinib is from about 10 mg to about 4000 mg. In some embodiment, dose of Afatinib is from 100 mg to 2000 mg. In some embodiment, dose of Afatinib is from about 1 mg to about 100 mg. In some embodiment, dose of Afatinib is from about 10 mg to about 50 mg. In some embodiments, this invention is a pharmaceutical composition or formulation comprising single dosage in the amount described above and at least one pharmaceutically acceptable excipient.

In some embodiments, for anti-aging effect Afatinib is administered intramuscularly. In some embodiments, the single dose for all age groups is 8-16 mg/kg body weight according to the following scheme: 16-31 kg body weight—250 mg Afatinib (½ ml); 32-46 kg body weight—500 mg Afatinib (1 ml); 47-62 kg body weight—500-750 mg Afatinib (1-1.5 ml); and over 63 kg body weight—750-1000 mg Afatinib (1.5-2 ml). In some embodiments, the dose can be repeated in 6-8 hours. Maximal daily dose—4.0 g Afatinib. In some embodiments, the dose in adults is 250-500 mg (½-1 tablet) 2 or 3 times daily. In some embodiments, maximal 24-hour dose is 3 g.

In some embodiments, Afatinib is administered at a dose of 250 mg, 2-3 times daily.

In some embodiments, Afatinib can be administered as a shot into the fatty part of the skin. In some embodiments, Afatinib can be administered as an infusion into a vein over a period of time.

In some embodiments Afatinib can be administered 75 mg/m2/day via subcutaneous injection or IV infusion for 7 days; In some embodiments repeat cycles every 4 weeks. In some embodiments Afatinib can be administered 100 mg/m2. In some embodiments Afatinib is administered from 5 mg/m2 to 200 mg/m2.

In some embodiments, compound selected from the group of EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any of its combination to achieve anti-aging effect can be administered by human or by other animal subject in a dosage and regimen to create or in some other embodiment to maintain a concentration of such compound in blood of such subject in 0.01 μM level, or in 0.03 μM level, or in 0.1 μM level, or in 0.02 μM level, or in 0.04 μM level, or in 0.08 μM level, or in 0.1 μM level, or in 0.2 μM level, or in 0.4 μM level, or in 0.8 μM, level, or in 1 μM level, or in 2 μM level, or in 4 μM level, or in 8 μM level, or in 10 μM level, or in 12 μM level, or in 14 μM level, or in 18 μM level, or in 20 μM level, or from 0.01 μM to 0.05 μM level, or from 0.01 μM to 0.1 μM level, or from 0.01 μM to 1 μM level, or from 0.1 μM to 0.1 μM level, or from 0.5 to 5 μM level, or from 0.5 to 5 μM level or in about level of +/−10% of the indicated above; in some embodiments such concentration should be maintained in blood for about 1 minute, or for about 10 minutes, or for about 1 hour, or for about 2 hours, or for about 4 hours, or for about 8 hours, or for about 16 hours, or for about 24 hours, or for about 48 hours, or for about 1 week, or for about 2 weeks, or for about 4 weeks, or for about 8 weeks, or for about 16 weeks, or for about 1 year, or for about 2 years, or for about 5 years, or lifelong; in some embodiments such concentration should be maintained in blood for about 10% of time, or for about 20% of time, or for about 30% of time, or for about 40% of time, or for about 50% of time, or for about 60% of time, or for about 70% of time, or for about 80% of time, or for about 90% of time, or for about 100% of time, or for about from 1% of time to 10% of time, or for about from 1% of time to about 90% of time, or for about from 10% of time to 50% of time, wherein time is selected from about 1 second, about 1 minute, or about 10 minutes, or about 1 hour, or about 2 hours, or about 4 hours, or about 8 hours, or about 16 hours, or about 24 hours, or about 48 hours, or about 1 week, or about 2 weeks, or about 4 weeks, or about 8 weeks, or about 16 weeks, or about 1 year, or about 2 years, or about 5 years, or from about 1 second to about 1 day, or from about 1 second to about 1 minute, or from about 1 minute to about 1 hour, or from about 1 hour to about 24 hours, or from about 24 hours to about 1 week, or from about 1 week to about 1 month, or from about 1 month to about 1 year, or from about 1 year to about 5 years, or lifelong.

In some embodiments, this invention is a delivery device or dosing device delivering compound selected from the group of EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any of its combination, and enabling maintaining a concentration of such compound in blood of such subject in 1 μM level at 30% of time, or from 0.5 to 5 μM level at from around 1% of time to around 100% of time, or from 0.5 to 5 μM level at from around 10% of time to around 50% of time or concentration described in this disclosure for a time described in this disclosure. A non-limiting example of such device—analog of insulin pump, automatically or semi-automatically or manually injecting compound selected from the group of EGFR inhibitor, AC480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any of its combination to maintain the a concentration of such compound in blood of such subject in 1 μM level at 30% of time, or from 0.5 to 5 μM level at from around 1% of time to around 100% of time, or from 0.5 to 5 μM level at from around 10% of time to around 50% of time or a concentration described in this disclosure for a time described in this disclosure.

In some embodiments, this invention is implantable medical device for controlled delivery of therapeutic agents of this disclosure. One of many examples of this invention can have a titanium reservoir, and a porous titanium oxide based membrane to control the rate of release of the therapeutic agent of this invention. The reservoir can contain a formulation of the active agent, including a stabilizer for the active agent, wherein the stabilizer is provided in an extended release configuration. Further details on some of the examples of devices for delivery of compounds of this invention to maintain the a concentration of such compound in blood of such subject in 1 μM level at 30% of time, or from 0.5 to 5 μM level at from around 1% of time to around 100% of time, or from 0.5 to 5 μM level at from around 10% of time to around 50% of time or a concentration described in this disclosure for a time described in this disclosure can be found e.g. in US application US20160220496A1.

In some embodiments, this invention is a formulation comprising compound selected from the group of EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any of its combination, enabling maintaining a concentration of such compound in blood of subject in 0, 5 μM 1 μM level at 30% of time, or from 0.5 μM to 5 μM level at from around 1% of time to around 100% of time, or from 0.5 μM to 5 μM level at from around 10% of time to around 50% of time or a concentration described in this disclosure for a time described in this disclosure.

In some embodiments, this invention is a slow release formulation or prodrug, comprising compound selected from the group of EGFR inhibitor, AC480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any of its combination, enabling maintaining a concentration of such compound in blood of subject in 1 μM level at 30% of time, or from 0.5 to 5 μM level at from around 1% of time to around 100% of time, or from 0.5 to 5 μM level at from around 10% of time to around 50% of time or a concentration described in this disclosure for a time described in this disclosure. In some embodiments, such slow release formulation or prodrug is for anti-aging use.

In some embodiments, this invention is a pharmaceutical composition for oral administration, including but not limited to tablet, capsule, suspension, drink etc., comprising Afatinib—100 mg, or 50 mg, or 75 mg, or from 10 mg to 150 mg, or from 20 mg to 100 mg, or from 1 mg to 150 mg. In some embodiments, this invention is a pharmaceutical composition, comprising Afatinib—100 mg, or 50 mg, or 75 mg, or from 10 mg to 150 mg, or from 20 mg to 100 mg, or from 1 mg to 150 mg, in some embodiments, this invention such pharmaceutical composition is for anti-aging use.

In some embodiments, Afatinib should be administered 40 mg orally, once daily. In case of Renal impairment: 30 mg orally, once daily in patients with severe renal impairment.

Afatinib should be administered once daily until disease progression or no longer tolerated by the patient.

In some embodiments, this invention is a pharmaceutical composition for oral administration, including but not limited to tablet, capsule, suspension, drink etc., comprising compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics, in some embodiments, this invention such pharmaceutical composition is for anti-aging use.

In some embodiments, this invention is a pharmaceutical composition and formulation, comprising amount of the agent selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics in the amount needed for a single dose to provide a concentration described in this disclosure for a time described in this disclosure calculated for a subject with weight of 50 kg or in some embodiments 75 kg or in some embodiments 90 kg and at least one pharmaceutically acceptable excipient.

Any of the compounds and combinations of this disclosure can be provided in formulations defined by the expert skilled in the art, including but not limited those known in the art and those suggested in the examples. In some embodiments, this invention the new use of compound selected from the group: EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 wherein a new use is a treatment of viral disease, decreasing a mortality of elderly and/or frail patients, decreasing a mortality of elderly and/or frail patients from viral disease, decreasing a mortality of elderly and/or frail patients from covid-19, decreasing a mortality of elderly and/or frail patients from influenza, decreasing a mortality of elderly and/or frail patients from disease, caused by SARS-CoV, SARS-CoV-2 (Severe acute respiratory syndrome coronavirus 2) or other coronavirus, reduction of pulmonary fibrosis caused by viral infection of SARS-CoV, reduction of pulmonary fibrosis caused by viral infection of SARS-CoV-2, reduction of virus titer of SARS-CoV, reduction of virus titer of SARS-CoV-2

In some embodiments, this invention the new use of compound selected from the group: EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 wherein a new use is a alleviation of symptoms of viral disease in elderly and/or frail patients, alleviation of symptoms of viral disease in elderly and/or frail patients, caused by covid-19, alleviation of symptoms of viral disease in elderly and/or frail patients, caused by influenza, alleviation of symptoms of viral disease in elderly and/or frail patients, of disease or condition, caused by SARS-CoV, SARS-CoV-2 (Severe acute respiratory syndrome coronavirus 2) or other coronavirus.

In some embodiments, this invention is at least one of the compound, composition or combination of this disclosure, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 useful for treatment and prevention of disease or condition selected from the group: accelerated aging associated with cancer treatment, cancer survival, cognitive impairment associated with cancer treatment and survival, trauma, stroke, HIV, Down syndrome, schizophrenia, other diseases and conditions associated with increased frailty.

In some embodiments, this invention is compound selected from EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 useful as vaccine adjuvant. Examples are influenze, SARS, SARS cov-2 and vaccines from other infections In some embodiment such vaccine adjuvant improves immune response to vaccine.

In some embodiments, this invention is described in the any one of the following items:

1. A method for treating a senescence associated disease or disorder in a subject comprising administering to the subject a compound or combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190; wherein the senescence associated disease or disorder is not a cancer, and wherein the such compound or combination is administered during a treatment course of 1-7 days every 0.5-12 months; provided that if the senescence associated disease or disorder is a senescence associated metabolic disorder, the senolytic combination is administered during a treatment course of 1-7 days every 4-12 months.
2. The method of item 1, wherein such compound or combination is administered once every 0.5-12 months; provided that if the senescence associated disease or disorder is a senescence associated metabolic disorder, the such compound or combination is administered once every 4-12 months.
3. The method of item 1, wherein the senescent cell-associated disease or disorder is a cardiovascular disease or disorder, inflammatory disease or disorder, a pulmonary disease or disorder, a neurological disease or disorder.
4. The method of item 3, wherein the cardiovascular disease or disorder is atherosclerosis.
5. The method of item 3, wherein the inflammatory disease or disorder is osteoarthritis.
6. The method of item 3, wherein the pulmonary disease or disorder is idiopathic pulmonary fibrosis or chronic obstructive pulmonary disease.
7. The method of item 3 wherein the neurological disease or disorder is selected from mild cognitive impairment; motor neuron dysfunction; Alzheimer's disease: Parkinson's disease; and macular degeneration.
8. The method of item 1, wherein the senescence associated metabolic disease or disorder is selected from diabetes, metabolic syndrome, and obesity.
9. The method of item 1, wherein the senescence-associated disease or disorder is a dermatological disease or disorder is selected from eczema, psoriasis, hyperpigmentation, nevi, rashes, atopic dermatitis, urticaria, diseases and disorders related to photosensitivity or photoaging, rhytides: pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas: and cutaneous lupus.
10. A method for treating a senescence-associated metabolic disease or disorder in a subject comprising administering to the subject a a compound or combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190, wherein the senolytic combination is administered during a treatment course of 1-7 days every 4-12 months, and wherein the metabolic disease or disorder is selected from diabetes, metabolic syndrome, and obesity.
11. The method of item 10, wherein the such compound or combination is administered once every 4-12 months.
12. The method of item 1 or item 10, wherein the senescent cell is selected from a senescent fibroblast, a senescent pre-adipocyte, a senescent epithelial cell, a senescent chondrocyte, a senescent neuron, and a senescent endothelial cell.
13. The method of item 10, wherein the senescent cell is a senescent pre-adipocyte

In some embodiments, this invention is described in the any one of the following items:

1. A pharmaceutical composition, comprising at least two compounds, selected from the group: EGFR inhibitor, AC480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount for anti-aging use.

2. A pharmaceutical composition, comprising from about 1 mg to about 5000 mg of active ingredient, wherein active ingredient is selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or their pharmaceutically acceptable salt or any combination of it or compounds or, optionally, having the similar SAR characteristics or any it's pharmaceutically acceptable salt, any it's hydrate, solvate, tautomer, geometric, optical and stereoisomer thereof, structurally related molecule, structural analog, functional analog, derivative, prodrug, or mixtures thereof in all ratios in therapeutically effective amount and at least one pharmaceutically acceptable excipient for anti-aging use.

3. A pharmaceutical composition of item 2, comprising from 10 mg to 100 mg of active ingredient.

4. A pharmaceutical composition of item 2, comprising from 1 mg to 50 mg of active ingredient.

5. A pharmaceutical composition of item 2, comprising from 1 mg to 75 mg of active ingredient.

6. A pharmaceutical composition of item 2, comprising from 1 mg to 100 mg of active ingredient.

7. A pharmaceutical composition of item 2, comprising from 1 mg to 100 mg of active ingredient.

8. A pharmaceutical composition of item 2, comprising from 1 mg to 25 mg of active ingredient.

9. A pharmaceutical composition of item 2, comprising from 1 mg to 10 mg of active ingredient.

10. A pharmaceutical composition of item 2, comprising from 1 mg to 5 mg of active ingredient.

11. A pharmaceutical composition, comprising from 1 mg to 99 mg of active ingredient, wherein active ingredient is selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 pharmaceutically acceptable salt, any it's hydrate, solvate, tautomer, geometric, optical and stereoisomer thereof, structurally related molecule, structural analog, functional analog, derivative, prodrug, or mixtures thereof in all ratios in therapeutically effective amount and at least one pharmaceutically acceptable excipient.

12. A pharmaceutical composition of item 11, comprising from 1 mg to 75 mg of active ingredient.

13. A pharmaceutical composition of item 11, comprising from 10 mg to 75 mg of active ingredient.

14. A pharmaceutical composition of item 11, comprising from 10 mg to 50 mg of active ingredient.

15. A pharmaceutical composition of item 11, comprising from 10 mg to 25 mg of active ingredient.

16. A pharmaceutical composition of item 11, comprising from 1 mg to 10 mg of active ingredient.

17. A pharmaceutical composition of any of items 1-16, wherein composition is for oral administration.

18. A pharmaceutical composition of any of items 1-16, wherein composition is for rectal administration.

19. A pharmaceutical composition of any of items 1-16, wherein composition is for injection.

20. A pharmaceutical composition of slow or controlled release providing a dosage according any of items 2-16.

21. A drug delivery device, providing dosage according any of items 2-16.

22. A use of pharmaceutical composition of any of the items 1-20 for anti-aging treatment.

23. A use of compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount for anti-aging treatment.

24. Kit, comprising pharmaceutical composition of any of the items 1-20 and instruction for using it as an anti-aging treatment.

25. Kit, comprising drug delivery device of item 21 and instruction for using it as in anti-aging treatment.

26. Kit, comprising compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount and instruction for its use in anti-aging treatment.

27. A use of compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount for manufacturing of anti-aging medication.

28. Method of anti-aging treatment, comprising administering to subject a therapeutically effective amount of compound, selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount or any combination of it.

29. A tangible medium comprising a computer program, which, when executed, causes a medium to perform a method comprising: attribution to the information about a subject an information about an anti-aging treatment related to compound, compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount or composition or combination comprising such compound.

30. A tangible medium comprising a computer program, which, when executed, causes a device to perform a method comprising: attribution to the information regarding compound, compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or any of their pharmaceutically acceptable salts, hydrates, solvates, tautomers, geometric, optical and stereoisomers thereof, structurally related molecules, structural analogs, functional analogs, derivatives, prodrugs, or mixtures thereof in all ratios in therapeutically effective amount or composition or combination comprising such compound, an information about use of such compound in anti-aging treatment.

31. Method, medium, use, composition of any of the items 22-30, wherein anti-aging treatment is a treatment or prevention an age-related disease or disorder or decline.

In some embodiments, this invention comprises the any one of the following items:

Item 1 A method of selectively killing one or more senescent cells in a subject in need thereof, the method comprising administering to the subject a composition comprising a compound selected from the group: EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190.
2. The method of item 1, wherein the compound that modulates FLIP is selected from the group consisting of droxinostat or a derivative thereof, piperlongumine or a derivative thereof and EF-24 or a derivative thereof.
4. The method of item 1, wherein the senescent cell is senescent due to replicative cellular senescence, premature cellular senescence, or therapy-induced senescence.
5. The method of item 1, wherein the senescent cell is from an age-related pathology.
6. The method of item 1, wherein the senescent cell is a therapy-induced senescent cell from normal and/or tumor tissue following DNA-damaging therapy.
7. The method of item 1, wherein selectively killing senescent cells is assessed by the LD50 of the composition, wherein the LD50 of the composition in non-senescent cells is greater than 3 times higher than the LD50 of the composition in senescent cells.
8. The method of item 7, wherein the killing is due to induction of apoptosis.
9. The method of item 7, wherein the killing is measured as a reduction in viable cells.
10. The method of item 9, wherein the reduction in viable cells is greater than 15%.
11. A method for delaying at least one feature of aging in a subject, the method comprising administering a composition comprising a therapeutically effective amount of a compound that modulates EGFR.
12. A method for anti-aging treatment, the method comprising administering a composition comprising a therapeutically effective amount of a compound that modulates EGFR
13. A method for delaying at least one feature of aging in a subject, the method comprising administering a composition comprising a therapeutically effective amount of a compound selected from the group: EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190
14. The method of any one of preceding items, wherein the subject has a received DNA-damaging therapy.
15. A method of treating an age-related disease or condition, the method comprising administering a composition comprising a therapeutically effective amount of compound that modulates EGFR, provided the age-related disease or condition is not cancer.
16. The method of item 15, wherein the compound that modulates EGFR is selected from the group consisting of EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or a derivative thereof.
17. The method of any one of preceding items, wherein the compound that modulates EGFR downregulates the nucleic acid or protein expression of EGFR.
18. The method of any one of preceding items, wherein the age-related disease or condition is a degenerative disease or a function-decreasing disorder.
19. The method of any one of preceding items, wherein the age-related disease or condition is due to DNA-damaging therapy.
20. The method of any one of preceding items, wherein the compound that modulates EGFR selectively kills therapy induced-senescent cells in normal and tumor tissues.
21. A method of treating a senescence-associated disease or condition, the method comprising administering a composition comprising a therapeutically effective amount of compound that modulates EGFR, provided the senescence-associated disease or condition is not cancer.
22. The method of any one of preceding items, wherein the compound that modulates EGFR is selected from the group consisting of EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or a derivative thereof.
23. The method of any one or preceding items, wherein the compound that modulates EGFR downregulates
24. any of the items of this application, wherein EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 is substituted by its pharmaceutically acceptable salt.

### EXAMPLES

### Example 1

### Example 1

1.1 Afatinib Treatment

C57BL/6J male mice aged 60 weeks (15 months) were ordered from Jackson Laboratories. Animals were allowed to acclimate to the housing environment (the animal holding room) for 3 days prior to the initiation of the study. Animals were housed individually to avoid fighting. The animals had ad libitum access to standard diet (2018, Global 18% Protein Rodent Diet from Envigo++++, San Diego, Calif.) and acidified water (pH 2.5-3.0) throughout the study period. The bedding material was hardwood chips (Sani-Chips, Cat #7115, Envigo++++, CA, USA) and was changed Bi-weekly.

Four days before treatment start blood samples were collected for CBC analysis. CBC test parameters (Table 1) were used for DFI calculation for each animal. Animals were stratified in 3 groups based on DFI: control (n=48), rapamycin (n=12), and afatinib (n=12). Thus, at the beginning of the experiment, there was no statistically significant difference between all groups.

Rapamycin was prepared as 40 mg/mL stock solution in DMSO and stored at −20 C. Stock solutions were diluted to 1.2 mg/ml. Final formulation—5% Tween-80, 5% PEG-400, 3% DMSO (from stock solution) in water

Afatinib was prepared as 66.7 mg/mL stock solution in DMSO and stored at −20 C. Stock solutions were diluted to 2 mg/ml. Final formulation—5% Tween-80, 5% PEG-400, 3% DMSO (from stock solution) in water.

Control animals were treated with 5% Tween-80, 5% PEG-400, 3% DMS (from stock solution) in water Test/Control Articles were administered as a daily (Mon-Sun) oral gavage dose

Animals' body weights was taken on Day-3 for randomization purposes and animals were monitored by weekly body weights trough out the entire study duration. Detailed clinical observations were performed weekly and recorded.

Every two weeks blood samples were collected and analyzed on hematology analyzer. The blood (120 μL) was collected into EDTA tubes via submandibular or facial vein using a lancet.

In Figure, we show the mean value of bioage in each treatment group.

TABLE 1
Complete blood counts parameters measured in our experiments,
which are used for calculation of marker of biological age.

| CBC parameter | Units |
|---|---|
| Granulocytes (neutrophils) | K/uL |
| Granulocytes | % |
| Hemoglobin | g/dL |
| Hematocrit | % |
| Lymphocytes | K/uL |
| Lymphocytes | % |
| Mean corpuscular hemoglobin | pg |
| Mean corpuscular hemoglobin concentration | g/dL |
| Mean corpuscular volume | fL |
| Platelets | K/uL |
| Red blood cells | M/uL |
| White blood cells | K/uL |

(a)

dFI correlates to age

Dynamical frailty index (dFI) as a function of age in the test experiments: MA0071 (males, orange diamonds), MA0071 (females, blue circles) and MA0072 (green triangles). The black curved dashed line is the exponential fit in the age groups younger than the average lifespan of NIH Swiss mice (indicated by the dashed vertical line). Red stars mark the average dFI in age-matched groups of frail animals from the MA0073 cohort. All data are presented as mean±SEM. dFI is associated with the number of senescent cells

Total flux (TF) in log scale representing p16-dependent luciferase reporter activity as a quantitative indicator of senescent cells: statistically significant correlations with age (a) and with dFI (b) in old mice (>50 weeks).

dFI is associated with the remaining lifespan

| | M (20 w) | F (20 w) | M (52 w) | F (52 w) | M (78 w) | F (78 w) |
|---|---|---|---|---|---|---|
| Cohort 1, | 98 | 125 | 155 | 188 | 167 | 150 |
| animals | | | | | | |
| dFI | −0.31 (1.68e−03) | −0.18 (4.77e−02) | −0.38 (1.35e−06) | −0.23 (1.37e−03) | −0.41 (5.12e−08) | −0.28 (5.23e−04) |
| Cohort 2, | 56 | 118 | 145 | 177 | 133 | 120 |
| animals | | | | | | |
| dFI | −0.31 (1.37e−03) | −0.21 (2.05e−02) | −0.38 (2.33e−06) | −0.25 (7.82e−04) | −0.32 (1.79e−04) | −0.31 (3.19e−04) |
| IGF1 | −0.31 (1.96e−03) | −0.17 (5.92e−02) | −0.10 (2.09e−03) | −0.11 (1.23e−03) | 0.03 (0.86e−01) | 0.05 (5.59e−01) |
| Body weight | −0.32 (1.01e−03) | −0.19 (3.84e−02) | −0.25 (2.25e−03) | −0.24 (9.71e−04) | 0.03 (0.92e−01) | 0.04 (0.78e−01) |

Spearman's rank-order correlation values and the corresponding p-values (in parentheses) for dFI with lifespan. Analysis is shown for two cohorts: Cohort 1 includes all animals with mortality data, Cohort 2 includes the subset of animals from Cohort 1 for which IGF1 measurements were available. Significant correlations (p<0.05) are highlighted in bold

dFI correlates with Physiological Frailty index

Correlation of dFI with the physiological frailty index (PFI) from “Identification of a blood test-based biomarker of aging through deep learning of aging trajectories in large phenotypic datasets of mice” Konstantin Avchaciov,1 Marina P. Antoch,2 Ekaterina L. Andrianova,3 Andrei E. Tarkhov,1, 4 Leonid I. Menshikov,1 Olga Burmistrova,1 Andrei V. Gudkov,3, 5 and Peter O. Fedichev1. available https://www.biorxiv.org/content/10.1101/2020.01.23.917286v1.full.pdf

here we measure Delta dFI

Drug==afatinib

### Example

1.2. Any of the compounds selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 can be tested the same way as shown in Example 1.1

In concentrations:

In concentrations effective in mice for their primary indication, In concentration 10 times less than their LD50,

In concentration selected from the following group: 100 times less than their LD50, 0.1 mg/mL, 0.01 mg/mL, 1 mg/mL, 10 mg/mL, 50 mg/mL, 100 mg/mL

Method of biological age/frailty index calculation that can be used to evaluate anti-aging effect is described in Identification of a blood test-based biomarker of aging through deep learning of aging trajectories in large phenotypic datasets of mice Konstantin Avchaciov,1 Marina P. Antoch,2 Ekaterina L. Andrianova,3 Andrei E. Tarkhov,1, 4 Leonid I. Menshikov,1 Olga Burmistrova,1 Andrei V. Gudkov,3, 5 and Peter O. Fedichev1

### Example 2

Prophetic composition comprising Exemplary Injection Formulation Containing an agent of this disclosure. The vial contains 5 mg of compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 as a powder for injection. Powder for injection is to be reconstituted with sterile water for injections and further diluted in 0.9% sodium chloride solution for infusion. After reconstitution, each vial contains substance for injection. Inactive ingredients: sodium phosphate monobasic monohydrate, sodium phosphate dibasic dihydrate, sucrose and polysorbate 80.

### Example 3

Prophetic Tablet Formulation Containing an agent of this disclosure.

A non-limiting example of a tablet containing agent selected from the group:

10 mg of compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 or its orally bioavailable form or delivery device or agent providing oral bioavailability, 50 mg microcrystalline cellulose, 10 mg potato or modified starch, 10 mg polyvinylpyrolidone, 10 mg methylcellulose, 25 mg dibasic calcium phosphate, 2 mg magnesium stearate, and 1.92 g Hydroxypropylmethylcellulose.

### Example 4

Injection Formulation

10 mg of compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 in a vial in a sterile form for reconstitution as a suspension for subcutaneous injection or reconstitution as a solution with further dilution for intravenous infusion and 100 mg of mannitol as a sterile lyophilized powder.

### Example 5

Prophetic Tablet Formulation

Compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190-20 mg, Ludipress—100 mg, Kollidon CL—10 mg, Magnesium stearate—10 mg, Aerosil—5 mg.

### Example 6

Prophetic Kit 1

Plastic box, comprising at least one oral tablet of the following composition:

Film-coated tablets 1 table.

Active Ingredient:

afatinib dimaleate 29.56/44.34/59.12/73.9 mg which corresponds to 20/30/40/50 mg of afatinib base, excipients: lactose monohydrate—123.86/185.79/247.72/309.65 mg; MCC—18.48/27.72/36.96/46.2 mg; colloidal anhydrous silicon dioxide—0.9/1.35/1.8/2.25 mg; crospovidone—3.6/5.4/7.2/9 mg; magnesium stearate—3.6/5.4/7.2/9 mg. film shell: hypromellose 2910—2.5/3.5/4/5 mg; macrogol 400—0.5/0.7/0.8/1 mg; titanium dioxide (E171)—1.2/0.6825/1.808/0.975 mg; talc—0.65/1.6625/1.04/2.375/mg; polysorbate 80—0.15/0.21/0.24/0.3 mg; dye indigocarmine aluminum varnish 11-14%—/0.245/0.112/0.35 mg

Tablets 20 mg: round biconvex with beveled edges, covered with a film shell from white to slightly yellowish color, the cross-section of the core is almost white.

Tablets 30 mg: round biconvex with beveled edges, covered with a blue film shell, the cross-section of the core is almost white.

Tablets 40 mg: round biconvex with beveled edges, covered with a film shell of blue color, the cross-section of the core is almost white.

Tablets 50 mg: oval biconvex, covered with a film shell of blue color, the cross-section of the core is almost white.

and paper instruction, wherein amongst other the following wording is present: “Afatinib is indicated for: anti-aging treatment, rejuvenation, frailty treatment, amelioration of moderate cognitive decline, amelioration hand grip strength lose and amelioration of other age related deficits or conditions”.

Prophetic Kit 2

Plastic box, comprising at least one oral tablet of example 5 or and paper instruction, wherein amongst other the following wording is present compound selected from the group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 is indicated for: anti-aging treatment, rejuvenation, frailty treatment, amelioration of moderate cognitive decline, amelioration hand grip strength lose and amelioration of other age related deficits”.

### Example 7

### Example 7.1

Prophetic Examples of Afatinib Use for Anti-Aging Treatment in Human.

As a prophetic examples the oral composition comprising Afatinib is administered orally every 12 hours lifelong, the dosage is calculated to get 1 mg of Afatinib per 1 kg of body weight.

As another prophetic example the same composition is administered orally every 12 hours for 1 week and is repeated when the biological age of the subject returns to the level when the first treatment had been administered.

As another prophetic example the same composition is administered orally every 12 hours for 1 month and is repeated when the biological age of the subject returns to the level when the first treatment had been administered.

As another prophetic example the same composition is administered orally every 12 hours for 6 months and is repeated when the biological age of the subject returns to the level when the first treatment had been administered.

As another prophetic example is any regimen as described above, wherein the dosage is calculated to get 0.5 mg of Afatinib per 1 kg of body weight.

As another prophetic example is any regimen as described above, wherein the dosage is calculated to get 0.2 mg of Afatinib per 1 kg of body weight.

As another prophetic example is any regimen as described above, wherein the dosage is calculated to get 0.05 mg of Afatinib per 1 kg of body weight.

One of many possible compositions (prophetic) for such regimen of administration for subject of 75 kg weight is a tablet formulation, comprising Afatinib 75 mg, Ludipress—17 mg, Kollidon CL—2 mg, Magnesium stearate—2 mg, Aerosil—1 mg.

The patient can be healthy, optionally aged subject or subject having at least one aging related disease, disorder or decline. For example, for such component of frailty as hands grip the hands grip force can be controlled by the means known in the art. For example, for cognitive decline such as test as Reaction time: Subject can complete a timed test of symbol matching, similar to the common card game ‘Snap’ hereafter referred to as reaction time (RT) (http://biobank.ctsu.ox.ac.uk/crystal/field.cgi?id=20023). The score on this task is the mean response time in milliseconds across trials which contained matching pairs. See for details e.g. Cognitive Test Scores in UK Biobank: Data Reduction in 480,416 Participants and Longitudinal Stability in 20,346 Participants (Donald M. Lyall et al., 2016). After the intervention the force of hands grip can be increased and the time of mean response time in test of symbol matching can be decreased as a marks of anti-aging effect of Afatinib. Afatinib can be used to prevent and treat frailty. A frailty index of the subject administering Afatinib can be measured by many ways known in the art, including but not limited to as described in (A global clinical measure of fitness and frailty in elderly people Rockwood et. Al 2005), (A standard procedure for creating a frailty index, Searle et al. 2008), (A Frailty Index Based On Deficit Accumulation Quantifies Mortality Risk in Humans and in Mice, K Rockwood et al.—2017).

Other anti-aging effects can be measured in many ways known in the art including but not limited to as described in “Frameworks for Proof-of-Concept Clinical Trials of Interventions That Target Fundamental Aging Processes”. Justice et al., 2016), an effect against particular diseases—by the methods, tests and equipment known to the expert in the particular disease etc. The list of the parameters that can be controlled after the intervention, as well as the regimen of such control (by default—1 month, 3 months, 6 months, 12 months, 18 months, 24 months, 5 years, 10 years, 20 years) after the treatment but can be changed), and methods and equipment for control are known to the experts in the field of specific aging relates disease, disorder or decline. Other parameters that can be improved by such administration are health span and lifespan.

### Example 7.2

Is the same as Example 7.1, wherein instead of Afatinib a compound selected from the following group is used: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190

### Example 8

Prophetic examples of compound selected from the following group: EGFR inhibitor, AC-480, AEE-788, AEE788, AFATINIB, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 use for anti-aging treatment in human.

In one prophetic example such compound is administered twice a day, as an injection subcutaneously, 50 mg/kg.

In another prophetic example such compound is administered twice a day, as an injection subcutaneously, 25 mg/kg.

In another prophetic example such compound is administered twice a day, as an injection subcutaneously, 10 mg/kg.

In another prophetic example such compound is administered twice a day, as an injection subcutaneously, 5 mg/kg.

In another prophetic example such compound is administered twice a day, as an injection subcutaneously, 2 mg/kg.

In one prophetic example such compound is administered in at least one of the dosages listed above once.

In another prophetic example such compound is administered in at least one of the dosages listed above during 1 week.

In another prophetic example such compound is administered in at least one of the dosages listed above during 1 month.

In another prophetic example such compound is administered in at least one of the dosages listed above during 6 months.

In another prophetic example such compound is administered in at least one of the dosages listed above during 12 months.

In another prophetic example the treatment is repeated when the biological age of the subject returns to the level when the first treatment had been administered.

The anti-aging effects can be measured but the methods known in the art, including but not limited as for Afatinib as described above.

### Example 9

### Example 9.1

Prophetic Mice Model of Metabolic Syndrome

Metabolic syndrome is a one of age related diseases. To confirm the effect of Afatinib on animal model of metabolic syndrome mice with diet induced obesity can be used. 15 months old C57BL/6J mice are divided into groups (n=10): one group receives standard laboratory chow (LabDiet JL 6% Oval, Cat #5K0Q), other groups are fed on 60% fat chow (DIO Rodent Purified Diet w/60% Energy From Fat, Cat #58Y1). Groups on high fat diet are treated with
1. vehicle
2. Afatinib, PO administration, every 6h 0.05 mg/kg
3. Afatinib, PO administration, every 6h 0.5 mg/kg
4. Afatinib, PO administration, every 6h 1 mg/kg
5. Afatinib, administration with food, compound should be mixed with feed at concentration 1 mg/kg,
6. Afatinib, administration with food, compound should be mixed with feed at concentration 5 mg/kg,
7. Afatinib, administration with food, compound should be mixed with feed at concentration 10 mg/kg,
8. Afatinib, administration with food, compound should be mixed with feed at concentration 20 mg/kg,
9. Afatinib, administration with food, compound should be mixed with feed at concentration 50 mg/kg
10. injection of Afatinib—0,025 mg/kg—2 times a day, IV
11. injection of Afatinib—0.25 mg/kg—2 times a day, IV
12. injection of Afatinib—0.01 mg/kg—2 times a day, IV
13. injection of Afatinib—0.5 mg/kg—2 times a day, IV
14. injection of Afatinib—1 mg/kg—2 times a day, IV
15. injection of Afatinib—2.5 mg/kg—2 times a day, IV
16. injection of Afatinib—5 mg/kg—2 times a day, IV
17. injection of Afatinib—10 mg/kg—2 times a day, IV
18. injection of Afatinib—25 mg/kg—2 times a day, IV
19. injection of Afatinib—50 mg/kg—2 times a day, IV
20. in other example, injection of Afatinib in the same dosage as shown above in this example is administered subcutaneously or orally

### Example 9.2

Prophetic Mice Model of Metabolic Syndrome

Metabolic syndrome is a one of age related diseases. To confirm the effect of compound selected from the group: EGFR inhibitor, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 on animal model of metabolic syndrome mice with diet induced obesity can be used. 15 months old C57BL/6J mice are divided into groups (n=10): one group receives standard laboratory chow (LabDiet JL 6% Oval, Cat #5K0Q), other groups are fed on 60% fat chow (DIO Rodent Purified Diet w/60% Energy From Fat, Cat #58Y1). Groups on high fat diet are treated with
1. vehicle
2. Compound selected from the group above, PO administration, every 24h 0.001 mg/kg
3. Compound selected from the group above, PO administration, every 24h 0.005 mg/kg
4. Compound selected from the group above, PO administration, every 24h 0.01 mg/kg
5. Compound selected from the group above, PO administration, every 24h 0.05 mg/kg
6. Compound selected from the group above, PO administration, every 24h 0.5 mg/kg
7. Compound selected from the group above, PO administration, every 24h 1 mg/kg
8. Compound selected from the group above, administration with food, compound should be mixed with feed at concentration 0.01 mg/kg,
9. Compound selected from the group above, administration with food, compound should be mixed with feed at concentration 0.1 mg/kg,
10. Compound selected from the group above, administration with food, compound should be mixed with feed at concentration 1 mg/kg,
11. Compound selected from the group above, administration with food, compound should be mixed with feed at concentration 5 mg/kg,
12. Compound selected from the group above, administration with food, compound should be mixed with feed at concentration 10 mg/kg,
13. Compound selected from the group above, administration with food, compound should be mixed with feed at concentration 20 mg/kg,
14. Compound selected from the group above, administration with food, compound should be mixed with feed at concentration 50 mg/kg
15. injection of Compound selected from the group above—0,025 mg/kg—1 time a day, IV
16. injection of Compound selected from the group above—0.25 mg/kg—1 time a day, IV
17. injection of Compound selected from the group above—0.01 mg/kg 1 time a day, IV
18. injection of Compound selected from the group above—0.5 mg/kg—1 time a day, IV
19. injection of Compound selected from the group above—1 mg/kg—1 time a day, IV
20. injection of Compound selected from the group above—2.5 mg/kg—1 time a day, IV
21. injection of Compound selected from the group above—5 mg/kg—1 time a day, IV
22. injection of Compound selected from the group above—10 mg/kg—1 time a day, IV
23. injection of Compound selected from the group above—25 mg/kg—1 time a day, IV
24. injection of Compound selected from the group above—50 mg/kg—1 time a day, IV
25. in other example, injection of Compound selected from the group above in the same dosage as shown above in this example is administered subcutaneously or orally

As other prophetic example of administration for anta-aging use is an oral administration or injection IV of combination of at least two of the compounds: selected from the group above in dosage selected for every compounds from dosages indicated above in this example or disclosure.

In one prophetic example the agent is administered as shown above for 1 day.

In another prophetic example the agent is administered as shown above for 1 week.

In another prophetic example the agent is administered as shown above for 2 weeks.

In another prophetic example the agent is administered as shown above for 4 weeks.

In another prophetic example the agent is administered as shown above for 8 weeks.

In another prophetic example the agent is administered as shown above for 16 weeks.

In another prophetic example the agent is administered as shown above for 32 weeks.

In another prophetic example the agent is administered as shown above lifelong.

After 6 weeks or after the end of treatment or at the time as defined by the expert in the field animal weight and fasting blood glucose, insulin, triglycerides, and leptin levels are assessed and glucose/insulin tolerance tests are performed. In prophetic example animals treated with the agent or combination of this disclosure will have healthier levels of weight, fasting blood glucose, insulin, triglycerides, leptin levels and glucose/insulin tolerance.

### Example 10

Prophetic Mice Experiment on Frailty and Lifespan

In this experiment the dosage and regimen as shown in example 9 or as defined as expert in the filed can be used. In prophetic test the agent and combinations of this disclosure should have an anti-aging effect. Effect of the treatment on biological age may be estimated based on changes in standard blood count [Gudkov], DNA methylation [Stubbs T M, Bonder M J, Stark A K, Krueger F; BI Ageing Clock Team, von Meyenn F, Stegle O, Reik W. Multi-tissue DNA methylation age predictor in mouse. Genome Biol. 2017 Apr. 11; 18(1):68; Horvath S. DNA methylation age of human tissues and cell types. Genome Biol. 2013; 14(10):R115], lifespan [Harrison D E, Strong R, Sharp Z D, Nelson J F, Astle C M, Flurkey K, Nadon N L, Wilkinson J E, Frenkel K, Carter C S, Pahor M, Javors M A, Fernandez E, Miller R A. Rapamycin fed late in life extends lifespan in genetically heterogeneous mice. Nature. 2009 Jul. 16; 460(7253):392-5], and healthspan assessed by frailty index. A frailty index is created by counting the accumulation of deficits in health across many systems in the body. Deficits measured to construct a frailty index include a large number of health-related variables related to the function of systems that are known to change with age in both human and animal models [Parks R J, Fares E, Macdonald J K, Ernst M C, Sinal C, Rockwood K, Howlett S E: A procedure for creating a frailty index based on deficit accumulation in aging mice. J Gerontol A Biol Sci Med Sci 2012; 67:217-227.]. These variables provide information about the following: (a) activity, including distance moved, velocity of movement and rearing frequency; (b) hemodynamic status, including heart rate, systolic and diastolic blood pressure; (c) body composition, including body mineral content, percent body fat and percent lean tissue; and (d) basic metabolism and organ function, including serum electrolyte levels, hematocrit and urea levels clinical signs, symptoms, diseases, and laboratory and radiographic abnormalities.

Cognitive score is assessed using Barnes maze, activity chamber and fear-conditioning tests Barnes maze test consists of a large circular maze containing 40 holes and elevated approximately 40 cm above the floor. The escape hole consists of a long PVC elbow joint connector that was similar in texture and color to the maze. The escape hole position is fixed within a day but changed for each successive day of testing. For each trial within a day, the starting location for the mouse was rotated relative to the escape hole position. Between each trial, the maze and escape hole were thoroughly cleaned to remove any cues that might affect performance in subsequent trials. With overhead illumination and a persistent 2 kHz tone, mice are given 90 s for each trial to identify the escape hole by jumping in or identifying the hole with extended/overt head pokes.

An activity chamber (Med Associates Inc., St. Albans, Vt.) is used to evaluate general locomotor activity and exploratory behavior in a novel environment. It consists of a square arena located in a sound-attenuated chamber. Mice are placed in the center of the arena and tracked by an automated tracking system with three planes of infrared detectors during a 10-min trial. Before each trial, the surface of the arena is cleaned with 10% ethanol. Distance moved, velocity, and rearing activity are measured.

To assess pain sensitivity to the foot-shock apparatus used for fear-conditioning each mouse is placed in the foot-shock chamber with a front-mounted camera to visualize various pain sensitivity parameters, which encompassed four clearly defined behaviors, ‘flinch’, ‘run’, ‘vocalization’, and ‘two-paw jump’. One-second shocks of various intensities are delivered each spaced by 30 s of recovery time. Thresholds reflected the minimum shock intensity at which each behavior was observed using the front-mounted camera video.

### Example 11

Prophetic Animal Experiment on Aging Related Diseases and Disorders

This prophetic experiment can be conducted on the relevant species (rat-by default but can be changed for other relevant species) and disease model of particular aging relates disease, disorder or decline, including but not limited to those described in this disclosure. The expert in the field can easily choose the relevant model for the particular aging relates disease, disorder or decline.

In this experiment the dosage and regimen as shown in example 9 or as defined as expert in the filed can be used.

The list of the parameters that should be controlled after the intervention, as well as the regimen of such control (by default—1 week, 1 months, 3 months, 6 months, 12 months, 18 months after the treatment by can be changed), and methods and equipment for control are known to the experts in the field of specific aging relates disease, disorder or decline.

### Example 12 Prophetic

Down-Regulation of EGFR Expression or EGFR Inhibition Selectively Kills Senescent Cells.

The IR-induced senescent (SC) WI38 cells and normal cells (NC) can after treated with vehicle (VEH) or EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 every of this compounds is in separate vial in concentration of 0.001 μM, 0.01 μM, 0.05 μM, 1 μM, 5 μM, 10 μM, 100 μM

IR-induced senescent (SC) WI38 cells can treated with vehicle (VEH) or any one of the compounds selected from the group above for 1 h or for 6 h or for 24 h.

Any one of the compounds selected from the group above selectively kills IR-induced senescent (SC) WI38 cells but has minimal effect on normal (NC) WI38 cells. Viable cells can be determined 72 h after normal (NC) and IR-induced senescent (SC) WI38 cells were treated with vehicle (VEH) or increasing concentrations of any one of the compounds selected from the group above. The data can be presented as a percentage of control cells treated with VEH.

### REFERENCES FOR THE EXAMPLES

1. Schimmer A D et al. Cancer Res. 2006: 66:2367-75.
2. Mawji I A et al. Cancer Res. 2007; 67:8307-15.
3. Shirley S & Micheau O. Cancer Letter 2013:332: 141-50.
4. Sanders Y Y et al. Redox Biol. 2013; 1:8-16.
5. Safa A R & Pollok K E. Cancer 2011: 3: 1639.71.
6. Raja S M et al. Mol Cancer Ther. 2008: 7:2212-23.
7. Lee S-J et al. Int J Oncol. 2011; 38:485492.
8. Siegelin M D et al. Neuroscie Lett. 2009; 453:92-7.
9. Chen S et al. Cancer Res. 2011; 71:6270-81.

### Example 13 Prophetic

In Vitro

To investigate anti-SARS-CoV-2 activity of compounds of this disclosure in vitro Vero E6 cells (ATCC-1586) are used. The cells are grown in Dulbecco's Modified Eagle Medium (DMEM) (Sigma Aldrich, Boston, Mass., USA) supplemented with 5% fetal bovine serum (Logan, Utah, USA) at 37° C. and 5% CO2. Cytotoxicity of the compounds in Vero cells is determined by the CCK8 assay.

For the treatment study Vero cells are seeded into 96-well plates at a density of 1x 104 cells/well and grown for 24 hours. Cells are then infected with SARS-CoV-2 at a multiplicity of infection (MOI) of 0.01 (100 PFU/well) for 2 hours at a temperature of 37° C. Virus input is washed with DMEM and the cells are then treated with medium containing test compounds at various concentrations (3-fold dilutions at seven different concentrations starting from CC50/10) for 24 or 48 hours.

For the prophylactic study Vero cells are grown as described above and are pretreated with various concentrations of the compounds ((3-fold dilutions at seven different concentrations starting from CC50/10) for 2 hours, after that drug containing medium is removed and SARS-CoV-2 virus-containing medium is added (as described for the treatment study) for 2 hours. Following this, the virus-containing medium is removed and replaced with fresh medium that does not contain drugs or viruses.

Efficacies are evaluated by quantification of viral copy numbers in the cell supernatant via quantitative real-time RT-PCR (qRT-PCR) (as described in [Huang C, Wang Y, Li X, et al. Clinical features of patients infected with 2019 novel coronavirus in Wuhan, China. Lancet. 2020. 395(10223): 497-506]) and confirmed with visualization of virus nucleoprotein (NP) expression through immunofluorescence microscopy at 48 h post infection.

Based on this data cytotoxicity (CC50) and virus replication and infection inhibition (EC50) is determined. EC50 is expected to be lower that CC50.

In Vivo

To investigate the antiviral activity of the compounds of this disclosure in vivo BALB/C mice are infected with SARS-CoV (MA15). All infections are performed in an animal biosafety level 3 facility using appropriate practices, including a HEPA-filtered bCON caging system, HEPA-filtered powered air-purifying respirators (PAPRs), and Tyvek suiting. All animals are grown to 10 weeks of age prior to use in experiments. The animals are anesthetized using a mixture of xylazine (0.38 mg/mouse) and ketamine (1.3 mg/mouse) in a 50-μl total volume by intraperitoneal injection. The mice are inoculated intranasally with 50 μl of either PBS or 1×105 PFU of rMA15 SARS-CoV, after which all animals are monitored daily for weight loss. Test compounds are administered every day via i.p route. Mice are euthanized at days 2, 5, or 9 postinfection, and lung tissue was harvested for further analysis.

Fifty percent tissue culture infectious dose (TCID50) values from SARS-CoV (MA15)-infected lungs is calculated by infecting multiple replicates of Vero E6 cells plated on 96-well plates. Serial dilutions (1:5) are performed for the virus-containing lung lysates in Vero E6 culture medium (complete MEM) such that at least the last two dilutions had no detectable virus in any of the replicates. The infection proceeds for 3 days before cells are fixed with 4% PFA and stained with 0.05% crystal violet in 20% methanol. The TCID50 is calculated using the formula log 10 TCID50=Xp+(0.5×D)−(D×Sp), where Xp is the last sample where all sample replicates are positive, D is the serial dilution log, and Sp is the sum of the proportion of replicates at all dilutions where positive values are seen (starting with the Xp dilution).

### Example 14 Prophetic Preclinical

Animals are treated with a dose corresponding to LD10 of chemotherapy agent. Frailty Index (FI) is measured as described in [Whitehead J C, Hildebrand B A, Sun M, Rockwood M R, Rose R A, Rockwood K, Howlett S E. A clinical frailty index in aging mice: comparisons with frailty index data in humans. J Gerontol A Biol Sci Med Sci. 2014; 69(6):621-32. Animals are left for one month of recovery. FI measurement is repeated. Animals are stratified in two groups: the control and the treatment groups. Treatment by compound selected from EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 is performed for 4-8 weeks followed by FI measurement, followed by another dose of LD10 of chemotherapy agent. Survival is evaluated. Animals in treatment group are expected to demonstrate better survival.

Clinical

Patients undergoing chemotherapy are enrolled. After the first round of chemotherapy FI is evaluated. Patients are stratified in placebo and treatment groups. Treatment groups receives compound selected from EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191 X, GW684626B, GW703087X, GW772405X, GW784684X, GW794726X, GW799251X, GW807930X, GW 869810X, HKI-357, IBRUTINIB, ICOTINIB, icotinib, LAPATINIB, lapatinib, LAPATINIB DITOSYLATE, LIPOPOLYSACCHARIDE E. COLI O55:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190. Patients receive the second round of chemotherapy treatment. Rate of withdrawal and dose reduction is evaluated. Patients in treatment group are expected to have lower rates of withdrawal and dose reduction.

The dose is used as shown in this application,

Example 14.2 The same dose is used as for primary indication.

Example 14.3 The same dose used is dose that is 2 times less, 5 times less or 10 times less as used as for primary indication.

Example 14.4 the following doses are used
1. vehicle
2. Compound selected from the group above, PO administration, every 24h 0.001 mg/kg
3. Compound selected from the group above, PO administration, every 24h 0.005 mg/kg
4. Compound selected from the group above, PO administration, every 24h 0.01 mg/kg
5. Compound selected from the group above, PO administration, every 24h 0.05 mg/kg
6. Compound selected from the group above, PO administration, every 24h 0.5 mg/kg
7. Compound selected from the group above, PO administration, every 24h 1 mg/kg
8. Compound selected from the group above, administration with food, compound should be mixed with feed at concentration 0.01 mg/kg,
9. Compound selected from the group above, administration with food, compound should be mixed with feed at concentration 0.1 mg/kg,
10. Compound selected from the group above, administration with food, compound should be mixed with feed at concentration 1 mg/kg,
11. Compound selected from the group above, administration with food, compound should be mixed with feed at concentration 5 mg/kg,
12. Compound selected from the group above, administration with food, compound should be mixed with feed at concentration 10 mg/kg,
13. Compound selected from the group above, administration with food, compound should be mixed with feed at concentration 20 mg/kg,
14. Compound selected from the group above, administration with food, compound should be mixed with feed at concentration 50 mg/kg
15. injection of Compound selected from the group above—0,025 mg/kg—1 time a day, IV
16. injection of Compound selected from the group above—0.25 mg/kg—1 time a day, IV
17. injection of Compound selected from the group above—0.01 mg/kg 1 time a day, IV
18. injection of Compound selected from the group above—0.5 mg/kg—1 time a day, IV
19. injection of Compound selected from the group above—1 mg/kg—1 time a day, IV
20. injection of Compound selected from the group above—2.5 mg/kg—1 time a day, IV
21. injection of Compound selected from the group above—5 mg/kg—1 time a day, IV
22. injection of Compound selected from the group above—10 mg/kg—1 time a day, IV
23. injection of Compound selected from the group above—25 mg/kg—1 time a day, IV
24. injection of Compound selected from the group above—50 mg/kg—1 time a day, IV
25. in other example, injection of Compound selected from the group above in the same dosage as shown above in this example is administered subcutaneously or orally

### Example 15

15.1 Prophetic Preclinical

Animals are treated with a dose corresponding to LD10 of chemotherapy agent. Frailty Index (FI) is measured as described in [Whitehead J C, Hildebrand B A, Sun M, Rockwood M R, Rose R A, Rockwood K, Howlett S E. A clinical frailty index in aging mice: comparisons with frailty index data in humans. J Gerontol A Biol Sci Med Sci. 2014; 69(6):621-32

]. Animals are left for one month of recovery. FI measurement is repeated. Animals are stratified in two groups: the control and the treatment groups. Treatment by afatinib in the dose from example 1 is performed for 4-8 weeks followed by FI measurement, followed by another dose of LD10 of chemotherapy agent. Survival is evaluated. Animals in treatment group are expected to demonstrate better survival.

Clinical

Patients undergoing chemotherapy are enrolled. After the first round of chemotherapy FI is evaluated. Patients are stratified in placebo and treatment groups. Treatment groups receives afatinib 20 mg and other group 40 mg daily orally. Patients receive the second round of chemotherapy treatment. Rate of withdrawal and dose reduction is evaluated. Patients in treatment group are expected to have lower rates of withdrawal and dose reduction.

15.2 the treatment is once a week

15.3 the treatment is once o month

15.4 the treatment lasts for 1 week or for 1 month

15.4 the treatment lasts from 1 day to 1 month

TABLE 0
Any of the item or embodiment of this disclosure may comprise
any one of the following molecules or drugs instead of other
egfr inhibitor. In some embodiments the respected drugs
can be used in the following dosages and regimens

| | | | | | |
|---|---|---|---|---|---|
| | | | Initial | | |
| Drug | | | Approval | Indi- | |
| (Trade name) | Class | Target | Date | cation | Dose |

| | | | | |
|---|---|---|---|---|
| Cetuximab | mAb | EGFR | | Intravenous |
| (Erbitux) | | | | 400 mg/m² |
| ImClone, Bristol- | | | | initial dose |
| Myers Squibb | | | | then 250 |
| | | | | mg/m² weekly |
| Erlotinib | TKI | EGFR | November | Oral |
| (Tarceva) | | | 2004 | 150 mg daily |
| OSI | | | | |
| Pharmaceuticals | | | | |
| Gefitinib | TKI | EGFR | | Oral |
| (Iressa) | | | | 250 mg daily |
| AstraZeneca | | | | |
| Lapatinib | TKI | EGFR/ | | Oral |
| (Tykerb) | | HER2 | | 1250 mg daily |
| SmithKline | | | | for 21 days |
| Beecham | | | | then 1 week |
| | | | | off |
| Panitumumab | mAb | EGFR | September | Intravenous |
| (Vectibix) | | | 2006 | 6 mg/kg every |
| Amgen | | | | 14 days |

| | | | | | | | Clinical |
|---|---|---|---|---|---|---|---|
| | | | | | | | Phase |
| Name | EGFR/ErbB1 | HER2/ErbB2 | ErbB3 | ErbB4 | mutant EGFR | Others | Clinical |
| Name | EGFR/ErbB1 | HER2/ErbB2 | ErbB3 | ErbB4 | mutant EGFR | Others | Phase |
| Erlotinib HC1 (OSI-744) | ++++ | | | | | | 4 |
| Gefitinib (ZD1839) | ++ | | | | | | 4 |
| Lapatinib (GW-572016) | ++ | +++ | | + | | | 4 |
| Ditosylate | | | | | | | |
| Afatinib (BIBW2992) | ++++ | ++ | | ++++ | | | 4 |
| Neratinib (HKI-272) | + | + | | | | KDR, Src | 3 |
| Canertinib (CI-1033) | ++++ | +++ | | | | | 2 |
| Lapatinib | ++ | +++ | | + | | | 4 |
| AG-490 (Tyrphostin | + | | | | | | |
| B42) | | | | | | | |
| CP-724714 | | ++ | | | | | 2 |
| Dacomitinib (PF299804, | +++ | + | | + | | | 3 |
| PF299, PF-00299804) | | | | | | | |
| WZ4002 | ++++ | | | | | | |
| Sapitinib (AZD8931) | +++ | +++ | +++ | | | | 2 |
| CUDC-101 | +++ | ++ | | | | HDAC, HDAC1, HDAC6 | 1 |
| AG-1478 (Tyrphostin | +++ | | | | | | |
| AG-1478) | | | | | | | |
| PD153035 HC1 | ++++ | | | | | | |
| Pelitinib (EKB-569) | + | + | | | | Src, MEK/ERK, Raf | 2 |
| AEE788 (NVP-AEE788) | ++++ | +++ | | + | | c-Abl, FLT1, c-Fms | 2 |
| AC480 (BMS-599626) | ++ | ++ | | + | | | 1 |
| AP26113-analog (ALK- | | | | | ++ | ALK, IGF1R, INSR | |
| IN-1) | | | | | | | |
| OSI-420 | ++++ | | | | | | |
| WZ3146 | ++++ | | | | | | |
| Allitinib (AST-1306) | ++++ | +++ | | ++++ | | | |
| Rociletinib (CO-1686, | ++ | | | | | | 3 |
| AVL-301) | | | | | | | |
| Varlitinib | +++ | ++++ | | | | | 3 |
| Icotinib | +++ | | | | | | 4 |
| TAK-285 | ++ | ++ | | + | | MEK1, Aurora B, LCK | 1 |
| WHI-P154 | +++ | | | | | Src, VEGFR, JAK3 | |
| Daphnetin | + | | | | | PKA, PKO | |
| PD168393 | ++++ | | | | | | |
| CNX-2006 | ++ | | | | ++ | | |
| Tyrphostin 9 | + | | | | | PDGFR | |
| AG-18 | + | | | | | | |
| PD153035 | ++++ | | | | | | |
| AG 555 | + | | | | | | |
| Theliatinib (HMPL-309) | +++ | | | | | EGFR T790M/L858R | |
| Avitinib (AC0010) | | | | | ++++ | JAK3, BTK | 3 |
| Lazertinib | ++++ | | | | ++++ | Dell9, L85R | 2 |
| (YH25448,GNS-1480) | | | | | | | |
| Gefitinib hydrochloride | ++ | | | | + | | |
| Cetuximab (anti-EGFR) | ++++ | | | | | | |
| Lifirafenib (BGB-283) | ++ | | | | + | WT A-RAF, C-RAF | 1 |
| | | | | | | (Y340/341D), BRAF | |
| | | | | | | (V600E) | |
| Nazartinib (EGF816, | ++ | | | | ++ | | 2 |
| NVS-816) | | | | | | | |
| Brigatinib (AP26113) | | | | | + | ALK, ROS1, FLT3 | 3 |
| AZD3759 | ++++ | | | | | | 3 |
| Afatinib (BIBW2992) | ++++ | ++ | | | | | 4 |
| Dimaleate | | | | | | | |
| Erlotinib | ++++ | | | | | | 4 |
| CL-387785 (EKI-785) | ++++ | | | | | | |
| Poziotinib (HM781-36B) | +++ | +++ | | ++ | | | 2 |
| Osimertinib (AZD9291) | ++ | | | | | | 3 |
| AZ5104 | ++++ | | | +++ | | ACK1, BLK, BRK | |
| HER2-Inhibitor-1 | | √ | | | | | |
| WZ8040 | √ | | | | | | |
| Genistein | √ | | | | | topo II | 4 |
| MTX-211 | √ | | | | | PI3K | |
| Naquotinib(ASP8273) | √ | | | | | | 3 |
| Olmutinib (HM61713, BI | √ | | | | | BTK | 2 |
| 1482694) | | | | | | | |
| Butein | √ | | | | | | |
| Chrysophanic Acid | √ | | | | | mTOR | |

Tables

TABLE 1
“Declines”.
Non-limiting list of parameters which age related change is
regarded as age related decline and which can be changed into
younger state or stabilized or its further change into the older
state delayed by anti-aging intervention of this disclosure

| Field | Units |
|---|---|
| Standing height | cm |
| Forced expiratory volume in 1-second (FEV1) | litres |
| Leg fat-free mass (right) | Kg |
| Leg predicted mass (right) | Kg |
| Basal metabolic rate | KJ |
| Forced vital capacity (FVC) | litres |
| Leg fat-free mass (left) | Kg |
| Leg predicted mass (left) | Kg |
| Systolic blood pressure, automated reading | mmHg |
| Heel bone mineral density (BMD) (left) | g/cm2 |
| Heel quantitative ultrasound index (QUI), direct | |
| entry (left) | |
| Whole body fat-free mass | Kg |
| Whole body water mass | Kg |
| Heel bone mineral density (BMD) T-score, | Std. Devs |
| automated (left) | |
| Speed of sound through heel (left) | m/s |
| Sitting height | cm |
| Heel bone mineral density (BMD) (right) | g/cm2 |
| Heel quantitative ultrasound index (QUI), direct | |
| entry (right) | |
| Speed of sound through heel (right) | m/s |
| Heel bone mineral density (BMD) T-score, | Std. Devs |
| automated (right) | |
| Peak expiratory flow (PEF) | litres/min |
| Leg fat percentage (left) | percent |
| Trunk fat-free mass | Kg |
| Leg fat percentage (right) | percent |
| Trunk predicted mass | Kg |
| Hand grip strength (left) | Kg |
| Heel broadband ultrasound attenuation (left) | dB/MHz |
| Heel broadband ultrasound attenuation (right) | dB/MHz |
| Hand grip strength (right) | Kg |
| Duration to first press of snap-button in each | milliseconds |
| round | |
| Mean time to correctly identify matches | milliseconds |
| Body fat percentage | percent |
| Trunk fat percentage | percent |
| Body mass index (BMI) | Kg/m2 |
| Leg fat mass (left) | Kg |
| Arm fat-free mass (left) | Kg |
| Arm predicted mass (left) | Kg |
| Arm fat-free mass (right) | Kg |
| Haematocrit percentage | percent |
| Arm predicted mass (right) | Kg |
| Waist circumference | cm |
| Leg fat mass (right) | Kg |
| Haemoglobin concentration | grams/decilitre |
| Arm fat percentage (left) | percent |
| Ankle spacing width (left) | mm |
| Whole body fat mass | Kg |
| Body mass index (BMI) | Kg/m2 |
| Pulse wave peak to peak time | milliseconds |
| Arm fat percentage (right) | percent |
| Weight | Kg |
| Mean corpuscular volume | femtolitres |
| Trunk fat mass | Kg |
| Pulse wave Arterial Stiffness index | |
| Ankle spacing width (right) | mm |
| Platelet crit | percent |
| Red blood cell (erythrocyte) count | 10\hat{ }12 cells/Litre |
| Mean sphered cell volume | femtolitres |
| Mean platelet (thrombocyte) volume | femtolitres |
| Weight | Kg |
| Arm fat mass (left) | Kg |
| Lymphocyte percentage | percent |
| Neutrophill percentage | percent |
| Arm fat mass (right) | Kg |
| Impedance of leg (left) | ohms |
| Mean reticulocyte volume | femtolitres |
| Platelet count | 10\hat{ }9 cells/Litre |
| Mean corpuscular haemoglobin | picograms |
| Impedance of leg (right) | ohms |
| Red blood cell (erythrocyte) distribution width | percent |
| Pulse rate, automated reading | bpm |
| Impedance of whole body | ohms |
| Diastolic blood pressure, automated reading | mmHg |
| Lymphocyte count | 10\hat{ }9 cells/Litre |
| Number of measurements made | |
| Neutrophill count | 10\hat{ }9 cells/Litre |
| Monocyte percentage | percent |
| Hip circumference | cm |
| Monocyte count | 10\hat{ }9 cells/Litre |
| Platelet distribution width | percent |
| Mean corpuscular haemoglobin concentration | grams/decilitre |
| Immature reticulocyte fraction | ratio |
| Impedance of arm (right) | ohms |
| Reticulocyte percentage | percent |
| Number of times snap-button pressed | |
| White blood cell (leukocyte) count | 10\hat{ }9 cells/Litre |
| Pulse rate | bpm |
| High light scatter reticulocyte count | 10\hat{ }12 cells/Litre |
| Basophill percentage | percent |
| Impedance of arm (left) | ohms |
| Pulse wave reflection index | |
| Eosinophill count | 10\hat{ }9 cells/Litre |
| Nucleated red blood cell count | 10\hat{ }9 cells/Litre |
| Eosinophill percentage | percent |
| Basophill count | 10\hat{ }9 cells/Litre |
| Reticulocyte count | 10\hat{ }12 cells/Litre |
| High light scatter reticulocyte percentage | percent |
| Nucleated red blood cell percentage | percent |

TABLE 2
Non-limiting list of biomarkers related to mortality risks

Red blood cell distribution width; Mean reticulocytes volume; Neutrophil number; Alpha-1-acid
glycoprotein; White blood cell count; Hemoglobin; Red blood cell count; Monocyte count; Basophil count;
Neutrophils percentage; Albumin, serum/plasma; Lymphocyte percentage; Hematocrit; Leukocyte
telomere length; Mean sphered cells volume; Very-low-density lipoprotein; Insulin-like growth factor 1;
Mean corpuscular hemoglobin; Mean corpuscular volume; Citrate; trans-lycopene; Monocyte percentage;
Platelet count; Reticulocytes count; Lymphocyte count; Platelet distribution width; Plateletcrit; Immature
reticulocytes fraction; Reticulocytes, high light scatter, percentage; Reticulocytes, high light scatter,
number; Reticulocytes percentage; Soluble CD14; Eosinophils percentage; 25-hydroxyvitamin D;
Adiponectin; Ascorbic acid; Brain natriuretic peptide; C-reactive protein; Cardiac troponin I; Estimated
glomerular filtration rate; Fibroblast growth factor-23; Gamma-glutamyltransferase; Glucose; Growth
differentiation factor 15; H-FABP; Homeostatic model assessment of insulin resistance; Homocysteine;
Lipoprotein-associated phospholipase A2 activity; N-terminal atrial natriuretic peptide; SUA; Type I
collagen degradation; Vitamin A; High-density lipoprotein cholesterol; Klotho; Leptin; Club (a.k.a. Clara)
cell secretory protein; Antinuclear autoantibodies; Soluble ST2; Alanine transaminase; Alkaline
phosphatase; Alpha-1-antichymotrypsin; Angiopoietin-2; ApoB/ApoA1 ratio; Asymmetric
dimethylarginine; C-reactive protein, high-sensitivity; Cardiac troponin T, high-sensitivity; Cholesterol;
Creatinine; Cystatin C; Fibrinogen; Glycated hemoglobin; Growth hormone; Homoarginine; Insulin-like
growth factor binding protein 1; Interleukin-6; Low-density lipoprotein cholesterol; Lycopene;
Mitochondrial DNA copy number; N-terminal pro-brain natriuretic peptide; Neutrophil gelatinase-
associated lipocalinin; Osteocalcin; Osteoprotegerin; Phosphorus; Testosterone; Triglycerides; Tumor
necrosis factor alpha; Uric acid; alpha-carotene; beta-trace protein; β2-microglobulin; Anion gap, serum
albumin adjusted; CD4:CD8 ratio; CD8 cells; Carboxyl-terminal telopeptide of collagen type I; Plasma
viscosity; Insulin-like growth factor binding protein 2; Peroxiredoxin 4; Stromal cell derived factor;
Carotenoids; Oxygenated carotenoids; Urea; sj/β-TREC ratio; Insulin-like growth factor binding protein 3;
Proinsulin; Factor VIIc; IgA to tissue transglutaminase; Bilirubin; Mean platelet volume; Galectin-3;
Interleukin-8; Loss of Y chromosome; Soluble tumour necrosis factor receptor 1; Symmetrical dimethyl
arginine; T cells; Thyroxine; Cell-free DNA concentration; beta-cryptoxanthin; Basophil percentage;
Interleukin-10; Apolipoprotein A-1; Amino-terminal propeptide of type I collagen; Estradiol-to-sex
hormone binding globulin ratio; Neutrophilia; Butyrylcholinesterase activity; Reticulocytes number;
Parathyroid hormone; Follicle stimulating hormone; Interleukin 1-beta; 17beta-E(2); Amino-terminal
peptide of procollagen type III;

The method of biological age/DFI that can be used to evaluate EGFR inhibitors for anti-aging use

1.-31. (canceled)

32. Method of anti-aging treatment in a subject comprising administering to the subject a compound or combination, combination, comprising such compound, modulating EGFR, including but not limited to EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN, CHLORIDE, DOCETAXEL, dovitinib, EBASTINE, EGF816, ERLOTINIB, erlotinib, ERLOTINIB, HYDROCHLORIDE, FALNIDAMOL, GEFITINIB, GENTIAN VIOLET, GSK-182497A, GSK-192082A, GSK-238063A, GSK-300014A, GSK-9786A, GW282974X, GW576484X, GW615311X, GW616030X, GW651576X, GW659893X, GW680191X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI 055:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190.

33. Method of claim 32, wherein method is a method of treating a senescence-associated disease or disorder, wherein the senescence-associated disease or disorder is not a cancer.

34. Method of claim 32, wherein method of anti-aging treatment is a treatment disease or disorder selected from the group: a cardiovascular disease or disorder, inflammatory disease or disorder, a pulmonary disease or disorder, a neurological disease or disorder, atherosclerosis, osteoarthritis, idiopathic pulmonary fibrosis, chronic obstructive pulmonary' disease, mild cognitive impairment: motor neuron dysfunction; Alzheimer's disease: Parkinson's disease; macular degeneration, diabetes, metabolic syndrome, obesity, dermatological disease or disorder, eczema, psoriasis, hyperpigmentation, nevi, rashes, atopic dermatitis, urticaria, photoaging, rhytides: pruritis: dysesthesia: eczematous eruptions: eosinophilic dermatosis; reactive neutrophilic dermatosis; pemphigus; pemphigoid; immunohullous dermatosis Tihrohistoeytic proliferations of skin: cutaneous lymphomas; cutaneous lupusm, therapy-induced senescence.

35. Method of claim 32, wherein method of anti-aging treatment is a treatment disease or disorder selected from the group: cardiovascular diseases, angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune disease, herniated intervertebral disc, inflammatory bowel disease, kyphosis, oral mucositis, lupus, interstitial cystitis, scleroderma, alopecia; neurodegenerative diseases, dementia, Huntington's disease, motor neuron dysfunction, age-related memory decline, depression/mood disorder; metabolic disease, diabetic ulcer, pulmonary diseases, age-related loss of pulmonary function, asthma, bronchiectasis, cystic fibrosis, emphysema, age-associated sleep apnea: gastrointestinal diseases such as Barrett's esophagus; liver fibrosis, muscle fatigue, oral submucosa fibrosis, pancreatic fibrosis, benign prostatic hyperplasia (BPH), and age-related sleep disorders: reproductive disorder, menopause (male and female), egg supply (female), sperm viability (male), fertility (male and female), sex drive, erectile function, arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, hyperpigmentation, immunobulious dermatosis, nevi, pemphigoid, pemphigus, pruritis, psoriasis, rashes, reactive neutrophilic dermatosis, rhytides, and urticarial; diabetic wound healing, post-transplant kidney fibrosis, carotid thrombosis.

36. A method of selectively killing one or more senescent cells in a subject in need thereof, the method comprising administering to the subject a composition comprising a compound selected from the group: EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-59%26, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB DAPH, DASATINIB, DELPHINIDIN CHLORIDE, DOCETAXEL, dovitinib, EBASTINE, EGF816, ERLOTINIB, erlotinib, ERLOTINIB HYDROCHLORIDE, FALNIDAMOL, GEFITINIB, GENTIAN VIOLET, GSK-182497A, GSK-192082A, GSK-238063A, GSK-300014A, GSK 969786A, GW282974X, GW576484X, GW615311X, GW616030X, GW651576X, GW659893X, GW680191X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI 055:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190.

37. Kit, comprising pharmaceutical composition, wherein such composition comprises compound selected from the group: EGFR inhibitor, small molecule EGFR inhibitor, EGFR antibody, EGFR humanized antibody, AC-480, AEE-788, AEE788, afatinib, ARRY-334543, AST-1306, AZD3759, AZD8931, BIBX-1382, BMS-599626, BMS-690514, BOSUTINIB, brigatinib, CADMIUM ACETATE, CADMIUM DICHLORIDE, CANERTINIB, canertinib, CANERTINIB DIHYDROCHLORIDE, CEP-32496, cetuximab, CGP-52421, CLOSANTEL, CP-724714, CUDC-101, DACOMITINIB, DAPH, DASATINIB, DELPHINIDIN, CHLORIDE, DOCETAXEL, dovitinib, EBASTINE, EGF816, ERLOTINIB, erlotinib, ERLOTINIB, HYDROCHLORIDE, FALNIDAMOL, GEFITINIB, GENTIAN VIOLET, GSK-182497A, GSK-192082A, GSK-238063A, GSK-300014A, GSK-969786A, GW282974X, GW576484X, GW615311X, GW616030X, GW651576X, GW659893X, GW680191X, GW684626B, GW703087X, GW772405X, GW784684X, GW794726X, GW799251X, GW807930X, GW869810X, HKI-357, IBRUTINIB, ICOTINIB, icotinib, LAPATINIB, lapatinib, LAPATINIB DITOSYLATE, LIPOPOLYSACCHARIDE E. COLI 055:B5, NAQUOTINIB, NERATINIB, olmutinib, osimertinib, panitumumab, PD-153035, PELITINIB, PKI-166, POZIOTINIB, PROSTRATIN, QUINAZOLINE, R-343, R-406, ROCILETINIB, salvianolic-acid-B, SAPITINIB, SORAFENIB, STAUROSPORINE, TAE-684, TAK-285, TANDUTINIB, TANZISERTIB, tucatinib, TYRPHOSTIN AG-1478, VANDETANIB, VARLITINIB, VATALANIB, XL-647, ZD-4190 and instruction for using it as an anti-aging treatment, but not for cancer treatment.
